(19) United States
(12) Patent Application Publication
(10) Pub. No.: US 2006/0293317 A1
(43) Pub. Date: Dec. 28, 2006

(54) Use of non-steroidal progesterone receptor modulators

(76) Inventors: Norbert Schmees, Berlin (DE); Wolfgang Schwede, Berlin (DE); Ulrike Fuhrmann, Berlin (DE); Anja Schmidt, Berlin (DE); Carsten Moeller, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/473,337

(22) Filed: Jun. 23, 2006

(30) Foreign Application Priority Data
Jun. 24, 2005 (DE) 102005030293.9

(51) Int. Cl. A61K 31/538; A61K 31/343

(52) U.S. Cl. 514/230.5; 514/469

(57) ABSTRACT

The present invention relates to the use of non-steroidal progesterone receptor modulators of the general formula (I) 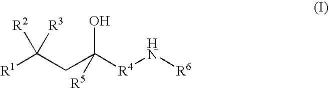 for the therapy and prophylaxis of gynaecological disorders and of hormone-dependent tumours, and for use for female fertility control and for hormone replacement therapy.

The present invention relates to the use of non-steroidal progesterone receptor modulators for the therapy and for prophylaxis of gynaecological disorders and of hormone-dependent tumours, and for use for female fertility control and for hormone replacement therapy.

The steroid hormone progesterone controls in a decisive manner the reproductive process in the female body. Progesterone is secreted in large quantities during the cycle and pregnancy respectively by the ovary and the placenta. Progesterone in cooperation with estrogens brings about cyclic changes in the uterine mucosa (endometrium) during the menstrual cycle. Elevated progesterone levels after ovulation influence the uterine mucosa to convert it into a state permitting nidation of an embryo (blastocyst). During pregnancy, progesterone controls the relaxation of the myometrium and maintains the function of the decidual tissue.

It is further known that progesterone inhibits endometrial proliferation by suppressing estrogen-mediated mitosis in uterine tissue (K. Chwalisz, R. M. Brenner, U. Fuhrmann, H. Hess-Stumpp, W. Elger, Steroids 65, 2000, 741-751).

Progesterone and progesterone receptors are also known to play a significant part in pathophysiological processes. Progesterone receptors have been detected in the foci of endometriosis, but also in tumours of the uterus, of the breast and of the CNS. It is further known that uterine leiomyomas grow progesterone-dependently.

The effects of progesterone in the tissues of the genital organs and in other tissues are achieved through interactions with progesterone receptors which are responsible for the cellular effects.

Progesterone receptor modulators are either pure agonists or inhibit the effect of progesterone partly or completely. Accordingly, substances are defined as pure agonists, partial agonists (SPRMs) and pure antagonists.

In accordance with ability of progesterone receptor modulators to influence the effect of the progesterone receptor, these compounds have a considerable potential as therapeutic agents for gynaecological and oncological indications and for obstetrics and fertility control.

Pure progesterone receptor antagonists completely inhibit the effect of progesterone on the progesterone receptor. They have anti-ovulatory properties and the ability to inhibit estrogen effects in the endometrium, as far as complete atrophy. They are therefore particularly suitable for intervening in the female reproductive process, e.g. post-ovulation, in order to prevent nidation, during pregnancy in order to increase the reactivity of the uterus to prostaglandins or oxytocin, or in order to achieve opening and softening (“ripening”) of the cervix, and in order to induce a great readiness of the myometrium to contract.

A beneficial effect on the pathological event is expected in foci of endometriosis and in tumour tissues which are equipped with progesterone receptors after administration of pure progesterone receptor antagonists. There might be particular advantages for influencing pathological states such as endometriosis or uterine leiomyomas if ovulation inhibition can additionally be achieved by the progesterone receptor antagonists. Ovulation inhibition also dispenses with some of the ovarian hormone production and thus the stimulating effect, deriving from this proportion, on the pathologically altered tissue.

The first progesterone receptor antagonist described, RU 486 (also mifepristone), was followed by a large number of analogues with progesterone receptor-antagonistic activity of varying strength. Whereas RU 486 shows an antiglucocorticoid effect in addition to the progesterone receptor-antagonistic effect, compounds synthesized later are notable in particular for a more selective effect as progesterone receptor antagonists.

Besides steroidal compounds such as onapristone or lilopristone, which are notable by comparison with RU 486 for a better dissociation of the progesterone receptor-antagonistic effect and the antiglucocorticoid effect, also known from the literature are various non-steroidal structures whose antagonistic effect on the progesterone receptor is being investigated [see, for example, S. A. Leonhardt and D. P. Edwards, Exp. Biol. Med. 227: 969-980 (2002) and R. Winneker, A. Fensome, J. E. Wrobel, Z. Zhang, P. Zhang, Seminars in Reproductive Medicine, Volume 23: 46-57 (2005)]. However, compounds disclosed to date have only moderate antagonistic activity compared with the known steroidal structures. The most effective non-steroidal compounds are reported to have in vitro activities which are 10% of the activity of RU 486.

The antiglucocorticoid activity is disadvantageous for therapeutic use, where the inhibition of progesterone receptors is at the forefront of the therapy. An antiglucocorticoid activity causes unwanted side effects at the dosages necessary for therapy. This may prevent administration of a therapeutically worthwhile dose or lead to discontinuation of the treatment.

Partial or complete reduction of the antiglucocorticoid properties is therefore an important precondition for therapy with progesterone receptor antagonists, especially for those indications requiring treatment lasting weeks or months.

In contrast to the pure antagonists, partial progesterone receptor agonists (SPRMs) show a residual agonistic property which may vary in strength. This leads to these substances showing potentially agonistic effects on the progesterone receptor in certain organ systems (D. DeManno, W. Elger, R. Garg, R. Lee, B. Schneider, H. Hess-Stumpp, G. Schuber, K. Chwalisz, Steroids 68, 2003, 1019-1032). Such an organ-specific and dissociated effect may be of therapeutic benefit for the described indications.

WO 03/059899 describes non-steroidal glucocorticoid mimetics or ligands of the general formula (I) 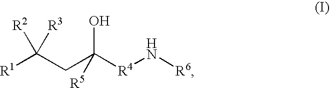
or their tautomers, prodrugs, solvates or salts.

These compounds, and pharmaceutical compositions comprising these compounds of the general formula (I), have a modulatory effect on the glucocorticoid receptor and are therefore suitable for the treatment of disorders mediated by the glucocorticoid receptor.

It is an object of the present invention to provide further non-steroidal progesterone receptor modulators. These compounds are intended to show a marked effect on the progesterone receptor and therefore be suitable for the therapy and prophylaxis of gynaecological disorders such as endometriosis, leiomyomas of the uterus, dysfunctional bleeding and dysmenorrhoea. In addition, the compounds of the invention are intended to be suitable for the therapy and prophylaxis of hormone-dependent tumours, for example of breast, endometrial, ovarian and prostate carcinomas. The compounds are further intended to be suitable for use in female fertility control and for female hormone replacement therapy.

The object is achieved according to the present invention by the use of the compounds of the general formula (I) 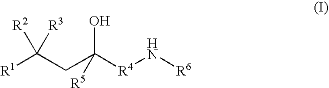
in which
R¹ is an aryl or heteroaryl group which is unsubstituted or optionally substituted by up to 3 radicals, where the substituents each independently of one another have the following meaning:
   C₁-C₅-alkyl, C₂-C₅-alkenyl, C₂-C₅-alkynyl, C₃-C₈-cycloalkyl, aryl, C₁-C₅-alkoxy, aryloxy, C₁-C₅-alkanoyl, aroyl, C₁-C₅-alkoxycarbonyl, C₁-C₅-alkanoyloxy, aminocarbonyloxy, C₁-C₅-alkylaminocarbonyloxy, C₁-C₅-dialkylaminocarbonyloxy, aminocarbonyl, C₁-C₅-alkylaminocarbonyl, C₁-C₅-dialkylaminocarbonyl, C₁-C₅-alkanoylamino, C₁-C₅-alkoxycarbonyl-amino, C₁-C₅-alkylsulphonylamino, C₁-C₅-alkylaminosulphonyl, C₁-C₅-dialkylaminosulphonyl, halogen, hydroxy, carboxy, cyano, trifluoromethyl, nitro, amino, with a mono- or disubstituted nitrogen atom, where the substituents on the nitrogen are each independently of one another
      C₁-C₅-alkyl or aryl or ureido, in which each nitrogen is substituted independently of the other by C₁-C₅-alkyl or C₁-C₅-alkylthio, where the sulphur may optionally be oxidized to a sulphoxide or sulphone,
   or in which each substituent may in turn be substituted independently of one another in each case by 1-3 radicals of the following meaning:
      methyl, halogen, hydroxy, oxo, cyano, trifluoromethyl and amino,
R² and R³ are each independently of one another hydrogen, C₁-C₅-alkyl, or
R² and R³ afford together with the C atom of the chain a ring having a total of 3-8 carbon atoms
R⁴ is CH₂ or C═O;
R⁵ is a carbocyclic, heterocyclic, aromatic or heteroaromatic ring which is attached directly or via a C₁-C₈-alkyl or a C₂-C₈-alkenyl and is unsubstituted or optionally substituted by 1-3 radicals, where each substituent in turn may be substituted independently of one another in each case by 1-3 radicals of the following meaning:
   C₁-C₅-alkyl, C₂-C₅-alkenyl, C₂-C₅-alkynyl, C₃-C₈-cycloalkyl, phenyl, C₁-C₅-alkoxy, phenoxy, C₁-C₅-alkanoyl, aroyl, C₁-C₅-alkoxycarbonyl, C₁-C₅-alkanoyloxy, aminocarbonyloxy, C₁-C₅-alkylaminocarbonyloxy, C₁-C₅-dialkylaminocarbonyloxy, aminocarbonyl, C₁-C₅-alkylaminocarbonyl, C₁-C₅-dialkylaminocarbonyl, C₁-C₅-alkanoylamino, C₁-C₅-alkoxycarbonylamino, C₁-C₅-alkylsulphonylamino, C₁-C₅-alkylaminosulphonyl, C₁-C₅-dialkylaminosulphonyl, halogen, hydroxy, carboxy, cyano, trifluoromethyl, nitro, amino, with a mono- or disubstituted nitrogen atom, where the substituents on the nitrogen are each independently of one another
      C₁-C₅-alkyl or aryl or ureido, in which each nitrogen is substituted independently of the other by C₁-C₅-alkyl or C₁-C₅-alkylthio, where the sulphur may optionally be oxidized to a sulphoxide or sulphone, and
R⁶ is an aromatic system which is unsubstituted or optionally substituted by 1-3 radicals, or is a group A or B: 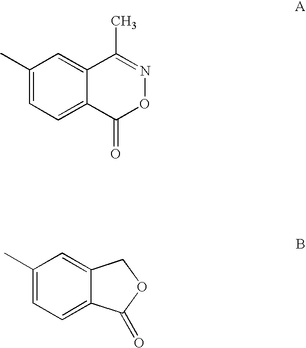
in which each substituent on R⁶ has independently of one another the following meaning:
   C₁-C₅-alkyl, C₂-C₅-alkenyl, C₂-C₅-alkynyl, C₃-C₈-cycloalkyl, C₁-C₅-alkoxy, C₁-C₅-alkanoyl, C₁-C₅-alkoxycarbonyl, C₁-C₅-alkanoyloxy, aminocarbonyloxy, C₁-C₅-alkylaminocarbonyloxy, C₁-C₅-dialkylaminocarbonyloxy, aminocarbonyl, C₁-C₅-alkylaminocarbonyl, C₁-C₅-dialkylaminocarbonyl, C₁-C₅-alkanoylamino, C₁-C₅-alkoxycarbonylamino, C₁-C₅-alkylsulphonylamino, C₁-C₅-alkylaminosulphonyl, C₁-C₅-dialkylaminosulphonyl, halogen, hydroxy, carboxy, cyano, trifluoromethyl, nitro, amino, with a mono- or disubstituted nitrogen atom, where the substituents on the nitrogen are each independently of one another
      C₁-C₅-alkyl or aryl or ureido, in which each nitrogen is substituted independently of the other by C₁-C₅-alkyl or C₁-C₅-alkylthio, where the sulphur may optionally be oxidized to a sulphoxide or sulphone,
         for the prophylaxis and therapy of gynaecological disorders such as endometriosis, leiomyomas of the uterus, dysfunctional bleeding and dysmenorrhoea, and for the prophylaxis and therapy of hormone-dependent tumours such as breast, endometrial, ovarian and prostate carcinomas and for use in female fertility control and hormone replacement therapy.

A further aspect of the invention relates to the use of the compounds of the general formula (I) in which the radical R¹ is a phenyl, naphthyl, indanyl, indenyl, chromanyl, dihydrobenzofuranyl, dihydroindolyl, dihydroquinolinyl, dihydroisoquinolinyl, tetrahydroquinolinyl, tetrahydroisoquinolinyl, thienyl, furanyl, pyrrolyl, pyridinyl, pyrimidinyl, pyrazinyl, indolyl, benzofuranyl or benzothienyl radical, where each of these radicals may in each case independently of one another be functionalized by 1 to 3 substituents, where each of these 1-3 substituents may independently of one another be C₁-C₃-alkyl, C₂-C₃-alkenyl, C₂-C₃-alkynyl, C₁-C₃-alkoxy, C₁-C₃-alkanoyl, C₁-C₃-alkanoylamino, halogen, hydroxy, cyano, trifluoromethyl, amino with a mono- or disubstituted nitrogen atom, where the substituents on the nitrogen are each independently of one another C₁-C₅-alkyl or aryl or ureido, in which each nitrogen is substituted independently of the other by C₁-C₅-alkyl or C₁-C₅-alkylthio, where the sulphur may optionally be oxidized to a sulphoxide or sulphone, and

in which each substituent may in turn be substituted independently of one another in each case by 1-3 radicals of the following meaning:

methyl, fluorine, chlorine, bromine, hydroxy, oxo, cyano, trifluoromethyl and amino, and

R² and R³ may independently of one another be C₁-C₃-alkyl, or

R² and R³ form together with the C atom of the chain a ring having a total of 3-8 carbon atoms;

R⁴ is CH₂ or C═O;

R⁵ is cyclopropyl, cyclobutyl, cyclopentyl, cyclohexyl, cyclopentylmethyl, cyclohexylmethyl, benzyl, cyclopentylethyl, cyclohexylethyl, phenethyl or phenyl-difluoromethyl, where each radical may be functionalized independently of one another by 1-3 substituents,

where each substituent of R⁵ is independently of one another methyl, methoxy, hydroxy, halogen, cyano or trifluoromethyl; and

R⁶ is a phenyl group which is optionally functionalized independently of one another by 1-3 substituents, or

R⁶ is a group A or B: 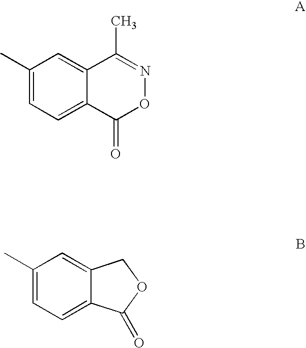
in which each substituent of R⁶ may independently of one another be methyl, methoxy, halogen, cyano or trifluoromethyl,
or a tautomer, a prodrug, solvate or salt thereof.

An additional aspect of the invention relates to the use of the compounds of the general formula (I) as described above with the following meaning of R⁶: aryl which is unsubstituted or optionally substituted by 1-3 radicals, where each substituent in R⁶ may be independently of one another C₁-C₅-alkyl, C₂-C₅-alkenyl, C₂-C₅-alkynyl, C₃-C₈-cycloalkyl, C₁-C₅-alkoxy, C₁-C₅-alkanoyl, C₁-C₅-alkoxycarbonyl, C₁-C₅-alkanoyloxy, C₁-C₅-alkylaminocarbonyloxy, C₁-C₅-dialkylaminocarbonyloxy, C₁-C₅-alkylaminocarbonyl, C₁-C₅-dialkylaminocarbonyl, C₁-C₅-alkanoylamino, C₁-C₅-alkoxycarbonylamino, C₁-C₅-alkylsulphonylamino, C₁-C₅-alkylaminosulphonyl, C₁-C₅-dialkylaminosulphonyl, halogen, hydroxy, carboxy, cyano, trifluoromethyl, nitro, amino with a mono- or disubstituted nitrogen atom, where the substituents on the nitrogen are each independently of one another C₁-C₅-alkyl or aryl or ureido, in which each nitrogen is substituted independently of the other by C₁-C₅-alkyl or C₁-C₅-alkylthio, where the sulphur may optionally be oxidized to a sulphoxide or sulphone.

The compounds of the general formula I may, owing to the presence of centres of asymmetry, exist as different stereoisomers. Both the use of the racemates and that of the separated stereoisomers belong to the subject-matter of the present invention.

A further aspect of the invention relates to the use of the compounds of the general formula (I) for producing a medicament for oral administration which comprises at least one compound of the general formula (I) as active ingredient in physiologically tolerated form and quantity.

It has been found that the non-steroidal compounds of the general formula (I) surprisingly show a noteworthy activity on the progesterone receptor. The compounds of the general formula (I) have strong antagonistic or strong partial agonistic effects on the progesterone receptor. The compounds of the general formula (I) are therefore suitable for the prophylaxis and therapy of gynaecological disorders such as endometriosis, leiomyomas of the uterus, dysfunctional bleeding and dysmenorrhoea. The compounds may furthermore be used for the treatment and prophylaxis of hormone-dependent tumours such as, for example, for breast, prostate and endometrial carcinoma.

The compounds according to the invention of the general formula (I) or their pharmaceutically acceptable salts are suitable for use for female fertility control or for female hormone replacement therapy.

The substituents which are defined as groups in the compounds according to the invention of the general formula I may in each case have the following meanings:

Alkyl means according to the invention a branched or unbranched saturated, monovalent aliphatic hydrocarbon chain. Examples thereof are methyl, ethyl, n-propyl, 1-methylethyl(isopropyl), n-butyl, n-pentyl, 1,1-dimethylethyl(tert-butyl).

Alkenyl means in the context of the invention a branched or unbranched unsaturated, monovalent aliphatic hydrocarbon chain having at least one carbon-carbon double bond. Preferred alkenyl radicals are ethenyl, propenyl, n-butenyl, isobutenyl, 3-methylbut-2-enyl, 12-pentenyl, heptenyl, octenyl, decenyl.

Alkynyl means in the context of the compounds according to the invention a branched or unbranched unsaturated, monovalent aliphatic hydrocarbon chain having at least one carbon-carbon triple bond. Examples which may be mentioned are ethynyl, propynyl, n-butynyl, 2-butynyl, 3-methylbutynyl, n-pentynyl, heptynyl, octynyl, decynyl.

Alkylene is intended to mean in the context of the invention branched or unbranched, divalent aliphatic hydrocarbon chains. Examples are methylene, ethylene, propylene, n-butylene and others. They may alternatively and equivalently also be called -(alkyl)-.

Alkenylene is intended to stand for a branched or unbranched, unsaturated, divalent aliphatic hydrocarbon chain having at least one carbon-carbon double bond. Mention may be made here in the context of the invention of ethenylene, propenylene, n-butenylene. They may also be called, alternatively and equivalently thereto, -(alkenyl)-.

Alkynylenes are intended to mean according to the invention branched or unbranched, unsaturated, divalent aliphatic hydrocarbon chains having at least one carbon-carbon triple bond. Ethynylene, propynylene, n-butynylene, 2-butynylene, 3-methylbutynylene, n-pentynylene, heptynylene, octynylene, decynylene may be mentioned as examples.

These radicals may also be called, alternatively and equivalently, -(alkynyl)-.

An alkoxy group is intended to mean in the context of the invention a monovalent radical of the formula AlkO— in which Alk is an alkyl group. Examples which may be mentioned are methoxy, ethoxy, propoxy, isopropoxy, butoxy, sec-butoxy, tert-butoxy, pentoxy.

Aryloxy is intended to stand for a monovalent radical of the formula ArO— in which Ar is an aryl. Phenoxy and naphthoxy may be mentioned in this connection.

Alkylcarbonyl or alkanoyl stands in the context of the invention for a monovalent radical of the formula AlkC(O)— in which Alk is an alkyl or hydrogen.

Arylcarbonyl or aroyl is intended to mean a monovalent radical of the formula ArC(O)— in which Ar is an aryl.

Acyl means a monovalent radical of the formula RC(O)— in which R is a substituent selected from hydrogen or an organic radical. Alkyl, aryl, arylalkyl, cycloalkyl, heterocyclyl, heteroaryl, heteroarylalkyl are preferred. Mention may likewise be made of alkylcarbonyl and arylcarbonyl.

Acylamino means in the context of the invention a monovalent radical of the formula RC(O)N(R)— in which R is a substituent selected from hydrogen or one of the abovementioned organic substituents.

Alkoxycarbonyl is regarded as being monovalent radicals of the formula AlkO—C(O)— in which Alk is alkyl. Examples which may be mentioned are methoxycarbonyl, ethoxycarbonyl, tert-butyloxycarbonyl.

Alkylcarbonyloxy or alkanoyloxy is intended to mean a monovalent radical of the formula AlkC(O)O— in which Alk is an alkyl radical.

Arylcarbonyloxy or aroyloxy means in the context of the invention a monovalent radical ArC(O)O— in which Ar is an aryl.

Alkylaminocarbonyloxy means in the context of the invention a monovalent radical R₂NC(O)O— in which each R is in each case independently of one another a hydrogen or a lower alkyl radical. A lower alkyl radical means in the context of the invention a C₁-C₈-alkyl radical.

Alkoxycarbonylamino means in the context of the invention a monovalent radical ROC(O)NH— in which R is a lower alkyl.

Alkylcarbonylamino or alkanoylamino means in the context of the invention a monovalent radical AlkC(O)NH— in which Alk is an alkyl radical. An alkylcarbonylamino radical also means for example an acetamido radical (CH₃C(O)NH—).

Alkylaminocarbonyloxy means in the context of the invention a monovalent radical AlkNHC(O)O— in which Alk is an alkyl radical.

Amino stands for an NH₂ group.

Alkylamino means in the context of the invention a monovalent radical (Alk)NH— in which Alk is an alkyl radical. Examples of an alkylamino group are methylamino, ethylamino, propylamino, butylamino, tert-butylamino.

Dialkylamino means in the context of the invention a monovalent radical (Alk)(Alk)N— in which each Alk is independently of the other an alkyl radical. Examples of a dialkylamino group are dimethylamino, methylethylamino, diethylamino, dipropylamino, ethylpropylamino.

A substituted amino group stands for a monovalent radical —NR₂ in which each R may be independently of the other a hydrogen or a substituent already mentioned hereinbefore, it not being possible for both radicals R to be simultaneously hydrogen. Examples which may be mentioned are alkyl, alkanoyl, aryl, arylalkyl, cycloalkyl, heterocyclyl, heteroaryl, heteroarylalkyl.

Alkoxycarbonylamino means in the context of the invention a monovalent radical AlkOC(O)NH— in which Alk is an alkyl radical.

Ureido means in the context of the invention a monovalent radical R₂NC(O)NH— in which each R is independently of the other a hydrogen or an alkyl radical.

Suitable for halogen are a fluorine, chlorine, bromine or iodine atom.

Halo means according to the invention that one or more hydrogen atoms are replaced by halogen.

Haloalkyl means a branched or unbranched saturated, monovalent aliphatic hydrocarbon chain in which one or more hydrogen atoms are replaced independently of one another by halogen atoms. Examples are chloromethyl, 1,2-dibromethyl, 1,1,1-trifluoropropyl, 2-iodobutyl, 1-chloro-2-bromo-3-fluoropentyl.

Alkylthio is intended to mean in the context of the invention a monovalent radical of the formula AlkS— in which Alk is an alkyl radical. Mention may preferably be made in this connection of methylthio, ethylthio, n-propylthio, isopropylthio, n-butylthio.

Sulphonyl means in the context of the invention a divalent radical —SO₂—.

Sulphonylamino means a divalent radical —SO₂NR— in which R may be a hydrogen atom or a substituent previously mentioned.

Aminosulphonyl means in the context of the invention a monovalent radical of the formula NR₂SO₂— in which R may be in each case independently of one another a hydrogen atom or a substituent previously mentioned.

Carbocycle means according to the invention a stable aliphatic 3-15-membered monocyclic or polycyclic, monovalent or divalent radical which consists exclusively of carbon atoms and hydrogen atoms and which comprises one or more rings connected or bridged together. 5-7-Membered monocyclic or 7-10-membered bicyclic rings are preferred in this connection. Unless mentioned otherwise, the carbocycle can be linked at any desired carbon atom provided that a stable structure is obtained. If the carbocyclic radical is substituted, this may be so at any desired carbon atom, once again provided that a stable structure is obtained. Examples thereof are cycloalkyl including a spirocycloalkyl radical, cycloalkylene, cycloalkenyl, cycloalkenylene, cycloalkynyl and cycloalkynylene.

Cycloalkyl means in the context of the invention a stable aliphatic 3-15-membered monocyclic or polycyclic, monovalent radical which consists exclusively of carbon atoms and hydrogen atoms and which comprises one or more rings connected or bridged together. 5-7-Membered monocyclic or 7-10-membered bicyclic rings are preferred in this connection. Unless mentioned otherwise, the cycloalkyl radical can be linked at any desired carbon atom provided that a stable structure is obtained. If the cycloalkyl radical is substituted, this may be so at any desired carbon atom, once again provided that a stable structure is obtained. Examples thereof are cyclopropyl, cyclobutyl, cyclopentyl, cyclohexyl, cycloheptyl, cyclooctyl, cyclononyl, cyclodecyl, norbornanyl, adamantyl, tetrahydronaphthyl (tetralinyl), 1-decalinyl, bicyclo[2.2.2]octanyl, 1-methylcyclopropyl, 2-methylcyclopentyl, 2-methylcyclooctyl.

Cycloalkenyl means in the context of the invention a stable aliphatic 5-15-membered monocyclic or polycyclic, monovalent radical having at least one carbon-carbon double bond, which consists exclusively of carbon atoms and hydrogen atoms and which comprises one or more rings connected or bridged together. 5-7-Membered monocyclic or 7-10-membered bicyclic rings are preferred in this connection. Unless mentioned otherwise, the cycloalkenyl radical can be linked at any desired carbon atom provided that a stable structure is obtained. If the cycloalkenyl radical is substituted, this may be so at any desired carbon atom, once again provided that a stable structure is obtained. Examples thereof are cyclopentenyl, cyclohexenyl, cycloheptenyl, cyclooctenyl, cyclononenyl, cyclodecenyl, norbornenyl, 2-methylcyclopentenyl, 2-methylcyclooctenyl.

Cycloalkynyl means in the context of the invention a stable aliphatic 8-15-membered monocyclic or polycyclic, monovalent radical having at least one carbon-carbon triple bond, which consists exclusively of carbon atoms and hydrogen atoms and which comprises one or more rings connected or bridged together. 8-10-Membered monocyclic or 12-15-membered bicyclic rings are preferred in this connection. Unless mentioned otherwise, the cycloalkynyl radical can be linked at any desired carbon atom provided that a stable structure is obtained. If the cycloalkynyl radical is substituted, this may be so at any desired carbon atom, once again provided that a stable structure is obtained. Examples thereof are cyclooctynyl, cyclononynyl, cyclodecynyl, 2-methylcyclooctynyl.

Cycloalkylene means in the context of the invention a stable aliphatic saturated 3-15-membered monocyclic or polycyclic, divalent radical which consists exclusively of carbon atoms and hydrogen atoms and which comprises one or more rings connected or bridged together. 5-7-Membered monocyclic or 7-10-membered bicyclic rings are preferred in this connection. Unless mentioned otherwise, the cycloalkylene radical can be linked at any desired carbon atom provided that a stable structure is obtained. If the cycloalkylene radical is substituted, this may be so at any desired carbon atom, once again provided that a stable structure is obtained. An example thereof is cyclopentylene.

Cycloalkenylene means in the context of the invention a stable aliphatic 5-15-membered monocyclic or polycyclic, divalent radical having at least one carbon-carbon double bond, which consists exclusively of carbon atoms and hydrogen atoms and which comprises one or more rings connected or bridged together. 5-7-Membered monocyclic or 7-10-membered bicyclic rings are preferred in this connection. Unless mentioned otherwise, the cycloalkenylene radical can be linked at any desired carbon atom provided that a stable structure is obtained. If the cycloalkenylene radical is substituted, this may be so at any desired carbon atom, once again provided that a stable structure is obtained. Examples thereof are cyclopentenylene, cyclohexenylene, cycloheptenylene, cyclooctenylene, cyclononenylene, cyclodecenylene, norbornenylene, 2-methylcyclopentenylene, 2-methylcyclooctenylene.

Cycloalkynylene means in the context of the invention a stable aliphatic 8-15-membered monocyclic or polycyclic, divalent radical having at least one carbon-carbon triple bond, which consists exclusively of carbon atoms and hydrogen atoms and which comprises one or more rings connected or bridged together. 8-10-Membered monocyclic or 12-15-membered bicyclic rings are preferred in this connection. Unless mentioned otherwise, the cycloalkynylene radical can be linked at any desired carbon atom provided that a stable structure is obtained. If the cycloalkynylene radical is substituted, this may be so at any desired carbon atom, once again provided that a stable structure is obtained. Examples thereof are cyclooctynylene, cyclononynylene, cyclodecynylene, 2-methylcyclooctynylene.

Aryl (Ar) means according to the invention an aromatic carbocyclic mono- or divalent monocyclic ring having 6-14 carbon atoms, for example phenyl or phenylene, or a fused ring system such as naphthyl or anthranyl. Unless mentioned otherwise, the aryl radical can be linked at any desired carbon atom provided that a stable structure is obtained. If the aryl radical is substituted, this may be so at any desired carbon atom, once again provided that a stable structure is obtained. Examples thereof are phenyl, naphthyl, anthryl, phenanthryl, indanyl, indenyl, biphenylyl.

Heteroaryl means in the context of the invention a stable aromatic 5-14-membered mono- or polycyclic monovalent or divalent radical which comprises one or more rings connected or bridged together. 5-7-Membered monocyclic or 7-10-membered bicyclic rings having 1-4 heteroatoms such as nitrogen, oxygen and sulphur are preferred in this connection, it being possible for the sulphur and the nitrogen each optionally to be oxidized or for the nitrogen to be a quaternary nitrogen. Unless mentioned otherwise, the heteroaryl radical can be linked at any desired heteroatom or carbon atom provided that a stable structure is obtained. If the heteroaryl radical is substituted, this may be so at any desired heteroatom or carbon atom, once again provided that a stable structure is obtained. Examples thereof are furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, triazinyl, indolizinyl, azaindolizinyl, indolyl, azaindolyl, diazaindolyl, dihydroindolyl, dihydroazaindoyl, isoindolyl, azaisoindolyl, benzofuranyl, furanopyridinyl, furanopyrimidinyl, furanopyrazinyl, furanopyridazinyl, dihydrobenzofuranyl, dihydrofuranopyridinyl, dihydrofuranopyrimidinyl, benzothienyl, thienopyridinyl, thienopyrimidinyl, thienopyrazinyl, thienopyridazinyl, dihydrobenzothienyl, dihydrothienopyridinyl, dihydrothienopyrimidinyl, indazolyl, azaindazolyl, diazaindazolyl, benzimidazolyl, imidazopyridinyl, benzthiazolyl, thiazolopyridinyl, thiazolopyrimidinyl, benzoxazolyl, oxazolopyridinyl, oxazolopyrimidinyl, benzisoxazolyl, purinyl, chromanyl, azachromanyl, quinolizinyl, quinolinyl, dihydroquinolinyl, tetrahydroquinolinyl, isoquinolinyl, dihydroisoquinolinyl, tetrahydroisoquinolinyl, cinnolinyl, azacinnolinyl, phthalazinyl, azaphthalazinyl, quinazolinyl, azaquinazolinyl, quinoxalinyl, azaquinoxalinyl, naphthyridinyl, dihydronaphthyridinyl, tetrahydronaphthyridinyl, pteridinyl, carbazolyl, acridinyl, phenazinyl, phenothiazinyl and phenoxazinyl.

Heterocycle means in the context of the invention a stable 5-14-membered mono- or polycyclic monovalent or divalent radical which comprises one or more rings connected or bridged together. 5-7-Membered monocyclic or 7-10-membered bicyclic rings having 1-3 heteroatoms such as nitrogen, oxygen and sulphur are preferred in this connection, it being possible for the sulphur and the nitrogen each optionally to be oxidized or for the nitrogen to be a quaternary nitrogen. Unless mentioned otherwise, the heterocycle can be linked at any desired heteroatom or carbon atom provided that a stable structure is obtained. If the heterocycle is substituted, this may be so at any desired heteroatom or carbon atom, once again provided that a stable structure is obtained. Examples thereof are pyrrolinyl, pyrrolidinyl, pyrazolinyl, pyrazolidinyl, piperidinyl, morpholinyl, thiomorpholinyl, piperazinyl, tetrahydropyranyl, tetrahydrothiopyranyl, tetrahydrofuranyl, hexahydropyrimidinyl, hexahydropyridazinyl.

The use of the compounds mentioned below for the prophylaxis and therapy of gynaecological disorders such as endometriosis, leiomyomatoses of the uterus, dysfunctional bleeding and dysmenorrhoea, and for the prophylaxis and therapy of hormone-dependent tumours such as breast, endometrial, ovarian and prostate carcinomas and for use in female fertility control and hormone replacement therapy is preferred according to the invention:
1) 2-Benzyl-2-hydroxy-4-methyl-4-phenylpentanoic acid (1-oxo-1,3-dihydroiso-benzofuran-5-yl)amide
2) 2-Hydroxy-4-methyl-2,4-diphenylpentanoic acid (1-oxo-1,3-dihydroisobenzo-furan-5-yl)amide
3) 2-Hydroxy-4-methyl-2-phenethyl-4-phenylpentanoic acid (1-oxo-1,3-dihydroiso-benzofuran-5-yl)amide
4) 2-Hydroxy-2-(3-methoxybenzyl)-4-methyl-4-phenylpentanoic acid (1-oxo-1,3-dihydroisobenzofuran-5-yl)amide
5) 2-Hydroxy-2-(4-methoxybenzyl)-4-methyl-4-phenylpentanoic acid (1-oxo-1,3-dihydroisobenzofuran-5-yl)amide
6) 2-Hydroxy-2-[2-(4-methoxyphenyl)ethyl]-4-methyl-4-phenylpentanoic acid (1-oxo-1,3-dihydroisobenzofuran-5-yl)amide
7) 2-Cyclohexylmethyl-2-hydroxy-4-methyl-4-phenylpentanoic acid (1-oxo-1,3-dihydroisobenzofuran-5-yl)amide
8) 2-(4-tert-Butylbenzyl)-2-hydroxy-4-methyl-4-phenylpentanoic acid (1-oxo-1,3-dihydroisobenzofuran-5-yl)amide
9) 2-Biphenyl-4-ylmethyl-2-hydroxy-4-methyl-4-phenylpentanoic acid (1-oxo-1,3-dihydroisobenzofuran-5-yl)amide
10) 2-Hydroxy-4-methyl-2-naphthalen-2-ylmethyl-4-phenylpentanoic acid (1-oxo-1,3-dihydroisobenzofuran-5-yl)amide
11) 2-Hydroxy-2-(3-hydroxybenzyl)-4-methyl-4-phenylpentanoic acid (1-oxo-1,3-dihydroisobenzofuran-5-yl)amide
12) 2-Hydroxy-4-methyl-2-(2-methyl-2-phenylpropyl)-4-phenylpentanoic acid (1-oxo-1,3-dihydroisobenzofuran-5-yl)amide
13) 2-Benzyl-4-(5-fluoro-2-methoxyphenyl)-2-hydroxy-4-methylpentanoic acid (1-oxo-1,3-dihydroisobenzofuran-5-yl)amide
14) 2-Cyclohexylmethyl-4-(5-fluoro-2-methoxyphenyl)-2-hydroxy-4-methylpentanoic acid (1-oxo-1,3-dihydroisobenzofuran-5-yl)amide
15) 2-Benzyl-4-(5-fluoro-2-hydroxyphenyl)-2-hydroxy-4-methylpentanoic acid (1-oxo-1,3-dihydroisobenzofuran-5-yl)amide
16) 2-Cyclohexylmethyl-4-(5-fluoro-2-hydroxyphenyl)-2-hydroxy-4-methylpentanoic acid (1-oxo-1,3-dihydroisobenzofuran-5-yl)amide
17) 4-(5-Fluoro-2-hydroxyphenyl)-2-hydroxy-4-methyl-2-(2-methyl-2-phenylpropyl)pentanoic acid (1-oxo-1,3-dihydroisobenzofuran-5-yl)amide
18) 2-(2-Chloro-6-fluorobenzyl)-4-(5-fluoro-2-methoxyphenyl)-2-hydroxy-4-methylpentanoic acid (1-oxo-1,3-dihydroisobenzofuran-5-yl)amide
19) 2-(3-Fluorobenzyl)-4-(5-fluoro-2-methoxyphenyl)-2-hydroxy-4-methylpentanoic acid (1-oxo-1,3-dihydroisobenzofuran-5-yl)amide
20) 2-(2-Fluorobenzyl)-4-(5-fluoro-2-methoxyphenyl)-2-hydroxy-4-methylpentanoic acid (1-oxo-1,3-dihydroisobenzofuran-5-yl)amide
21) 2-(3,4-Difluorobenzyl)-4-(5-fluoro-2-methoxyphenyl)-2-hydroxy-4-methylpentanoic acid (1-oxo-1,3-dihydroisobenzofuran-5-yl)amide
22) 2-(2-Chloro-6-fluorobenzyl)-4-(5-fluoro-2-hydroxyphenyl)-2-hydroxy-4-methylpentanoic acid (1-oxo-1,3-dihydroisobenzofuran-5-yl)amide
23) 2-(3-Fluorobenzyl)-4-(5-fluoro-2-hydroxyphenyl)-2-hydroxy-4-methylpentanoic acid (1-oxo-1,3-dihydroisobenzofuran-5-yl)amide
24) 2-(2-Fluorobenzyl)-4-(5-fluoro-2-hydroxyphenyl)-2-hydroxy-4-methylpentanoic acid (1-oxo-1,3-dihydroisobenzofuran-5-yl)amide
25) 2-(3,4-Difluorobenzyl)-4-(5-fluoro-2-hydroxyphenyl)-2-hydroxy-4-methylpentanoic acid (1-oxo-1,3-dihydroisobenzofuran-5-yl)amide
26) 2-(4-Fluorobenzyl)-4-(5-fluoro-2-methoxyphenyl)-2-hydroxy-4-methylpentanoic acid (1-oxo-1,3-dihydroisobenzofuran-5-yl)amide
27) 4-(5-Fluoro-2-methoxyphenyl)-2-hydroxy-4-methyl-2-(3-methylbenzyl)pentanoic acid (1-oxo-1,3-dihydroisobenzofuran-5-yl)amide
28) 2-(4-Fluorobenzyl)-4-(5-fluoro-2-hydroxyphenyl)-2-hydroxy-4-methylpentanoic acid (1-oxo-1,3-dihydroisobenzofuran-5-yl)amide
29) 4-(5-Fluoro-2-hydroxyphenyl)-2-hydroxy-4-methyl-2-(3-methylbenzyl)pentanoic acid (1-oxo-1,3-dihydroisobenzofuran-5-yl)amide
30) 2-(3,5-Difluorophenyl)-4-(5-fluoro-2-hydroxyphenyl)-2-hydroxy-4-methylpentanoic acid (1-oxo-1,3-dihydroisobenzofuran-5-yl)amide
31) 2-Benzyl-4-(5-fluoro-2-methoxyphenyl)-2-hydroxy-4-methylpentanoic acid (4-methyl-1-oxo-1H-benzo[d][1,2]oxazin-6-yl)amide
32) 2-Benzyl-4-(5-fluoro-2-hydroxyphenyl)-2-hydroxy-4-methylpentanoic acid (4-methyl-1-oxo-1H-benzo[d][1,2]oxazin-6-yl)amide
33) 2-Cyclohexylmethyl-4-(5-fluoro-2-methoxyphenyl)-2-hydroxy-4-methylpentanoic acid (4-methyl-1-oxo-1H-benzo[d][1,2]oxazin-6-yl)amide
34) 2-Benzyl-4-(5-fluoro-2-methoxyphenyl)-2-hydroxy-4-methylpentanoic acid (3,5-dichlorophenyl)amide
35) 4-(5-Fluoro-2-methoxyphenyl)-2-hydroxy-4-methyl-2-(2-methylbenzyl)pentanoic acid (1-oxo-1,3-dihydroisobenzofuran-5-yl)amide
36) 2-(3,5-Dimethylbenzyl)-4-(5-fluoro-2-methoxyphenyl)-2-hydroxy-4-methylpentanoic acid (1-oxo-1,3-dihydroisobenzofuran-5-yl)amide
37) 2-(2,5-Difluorobenzyl)-4-(5-fluoro-2-methoxyphenyl)-2-hydroxy-4-methylpentanoic acid (1-oxo-1,3-dihydroisobenzofuran-5-yl)amide
38) 2-(2,5-Difluorobenzyl)-4-(5-fluoro-2-hydroxyphenyl)-2-hydroxy-4-methylpentanoic acid (1-oxo-1,3-dihydroisobenzofuran-5-yl)amide
39) 4-(5-Fluoro-2-hydroxyphenyl)-2-hydroxy-4-methyl-2-(2-methylbenzyl)pentanoic acid (1-oxo-1,3-dihydroisobenzofuran-5-yl)amide
40) 2-(3,5-Dimethylbenzyl)-4-(5-fluoro-2-hydroxyphenyl)-2-hydroxy-4-methylpentanoic acid (1-oxo-1,3-dihydroisobenzofuran-5-yl)amide
41) 2-Cyclohexylmethyl-4-(5-fluoro-2-methoxyphenyl)-2-hydroxy-4-methylpentanoic acid (3,5-dichlorophenyl)amide
42) 2-Cyclohexylmethyl-4-(5-fluoro-2-hydroxyphenyl)-2-hydroxy-4-methylpentanoic acid (4-methyl-1-oxo-1H-benzo[d][1,2]oxazin-6-yl)amide
43) 2-Benzyl-4-(5-fluoro-2-hydroxyphenyl)-2-hydroxy-4-methylpentanoic acid (3,5-dichlorophenyl)amide
44) 2-Cyclohexylmethyl-4-(5-fluoro-2-hydroxyphenyl)-2-hydroxy-4-methylpentanoic acid (3,5-dichlorophenyl)amide
45) 2-Benzyl-4-(5-fluoro-2-methoxyphenyl)-2-hydroxy-4-methylpentanoic acid phenylamide
46) 2-Cyclohexylmethyl-4-(5-fluoro-2-methoxyphenyl)-2-hydroxy-4-methylpentanoic acid phenylamide
47) 2-(3-Chlorobenzyl)-4-(5-fluoro-2-hydroxyphenyl)-2-hydroxy-4-methylpentanoic acid (1-oxo-1,3-dihydroisobenzofuran-5-yl)amide
48) 4-(5-Fluoro-2-methoxyphenyl)-2-hydroxy-2-[2-(4-methoxyphenyl)ethyl]-4-methylpentanoic acid (1-oxo-1,3-dihydroisobenzofuran-5-yl)amide
49) 4-(5-Fluoro-2-methoxyphenyl)-2-hydroxy-2-(2-methoxybenzyl)-4-methylpentanoic acid (1-oxo-1,3-dihydroisobenzofuran-5-yl)amide
50) 2-Benzyl-4-(5-fluoro-2-hydroxyphenyl)-2-hydroxy-4-methylpentanoic acid phenylamide
51) 2-Cyclohexylmethyl-4-(5-fluoro-2-hydroxyphenyl)-2-hydroxy-4-methylpentanoic acid phenylamide
52) 4-(5-Fluoro-2-methoxyphenyl)-2-hydroxy-4-methyl-2-phenethylpentanoic acid (1-oxo-1,3-dihydroisobenzofuran-5-yl)amide
53) 2-(2-Chlorobenzyl)-4-(5-fluoro-2-methoxyphenyl)-2-hydroxy-4-methylpentanoic acid (1-oxo-1,3-dihydroisobenzofuran-5-yl)amide
54) 4-(5-Fluoro-2-hydroxyphenyl)-2-hydroxy-4-methyl-2-phenethylpentanoic acid (1-oxo-1,3-dihydroisobenzofuran-5-yl)amide
55) 4-(5-Fluoro-2-hydroxyphenyl)-2-hydroxy-2-[2-(4-hydroxyphenyl)ethyl]-4-methylpentanoic acid (1-oxo-1,3-dihydroisobenzofuran-5-yl)amide
56) 2-(2-Chlorobenzyl)-4-(5-fluoro-2-hydroxyphenyl)-2-hydroxy-4-methylpentanoic acid (1-oxo-1,3-dihydroisobenzofuran-5-yl)amide
57) 4-(5-Fluoro-2-hydroxyphenyl)-2-hydroxy-2-(2-hydroxybenzyl)-4-methylpentanoic acid (1-oxo-1,3-dihydroisobenzofuran-5-yl)amide
58) 2-(2-Bromobenzyl)-4-(5-fluoro-2-methoxyphenyl)-2-hydroxy-4-methylpentanoic acid (1-oxo-1,3-dihydroisobenzofuran-5-yl)amide
59) 2-(2-Bromobenzyl)-4-(5-fluoro-2-hydroxyphenyl)-22-hydroxy-4-methylpentanoic acid (1-oxo-1,3-dihydroisobenzofuran-5-yl)amide
60) 2-Benzyl-4-(5-fluoro-2-methoxyphenyl)-2-hydroxy-4-methylpentanoic acid (3,5-dimethylphenyl)amide
61) 2-Cyclohexylmethyl-4-(5-fluoro-2-methoxyphenyl)-2-hydroxy-4-methylpentanoic acid (3,5-dimethylphenyl)amide
62) 2-Benzyl-4-(5-fluoro-2-methoxyphenyl)-2-hydroxy-4-methylpentanoic acid (3,5-bistrifluoromethylphenyl)amide
63) 2-Cyclohexylmethyl-4-(5-fluoro-2-methoxyphenyl)-2-hydroxy-4-methylpentanoic acid (3,5-bistrifluoromethylphenyl)amide
64) 2-Benzyl-4-(5-fluoro-2-methoxyphenyl)-2-hydroxy-4-methylpentanoic acid (3,5-dimethoxyphenyl)amide
65) 2-Cyclohexylmethyl-4-(5-fluoro-2-methoxyphenyl)-2-hydroxy-4-methylpentanoic acid (3,5-dimethoxyphenyl)amide
66) 2-Benzyl-4-(5-fluoro-2-hydroxyphenyl)-2-hydroxy-4-methylpentanoic acid (3,5-dimethylphenyl)amide
67) 2-Cyclohexylmethyl-4-(5-fluoro-2-hydroxyphenyl)-2-hydroxy-4-methylpentanoic acid (3,5-dimethylphenyl)amide
68) 2-Benzyl-4-(5-fluoro-2-hydroxyphenyl)-2-hydroxy-4-methylpentanoic acid (3,5-bistrifluoromethylphenyl)amide
69) 2-Cyclohexylmethyl-4-(5-fluoro-2-hydroxyphenyl)-2-hydroxy-4-methylpentanoic acid (3,5-bistrifluoromethylphenyl)amide
70) 2-Benzyl-4-(5-fluoro-2-hydroxyphenyl)-2-hydroxy-4-methylpentanoic acid (3,5-dihydroxyphenyl)amide
71) 2-Cyclohexylmethyl-4-(5-fluoro-2-hydroxyphenyl)-2-hydroxy-4-methylpentanoic acid (3,5-dihydroxyphenyl)amide
72) 2-(5-Fluoro-2-methoxybenzyl)-2-hydroxy-4-methyl-4-phenylpentanoic acid (1-oxo-1,3-dihydroisobenzofuran-5-yl)amide
73) 2-(5-Fluoro-2-hydroxybenzyl)-2-hydroxy-4-methyl-4-phenylpentanoic acid (1-oxo-1,3-dihydroisobenzofuran-5-yl)amide
74) 2-(5-Fluoro-2-methoxybenzyl)-4-(5-fluoro-2-methoxyphenyl)-2-hydroxy-4-methylpentanoic acid (1-oxo-1,3-dihydroisobenzofuran-5-yl)amide
75) 2-(5-Fluoro-2-hydroxybenzyl)-4-(5-fluoro-2-hydroxyphenyl)-2-hydroxy-4-methylpentanoic acid (1-oxo-1,3-dihydroisobenzofuran-5-yl)amide
76) 2-(3,5-Dimethoxybenzyl)-2-hydroxy-4-methyl-4-phenylpentanoic acid (1-oxo-1,3-dihydroisobenzofuran-5-yl)amide
77) 2-(3,5-Dihydroxybenzyl)-2-hydroxy-4-methyl-4-phenylpentanoic acid (1-oxo-1,3-dihydroisobenzofuran-5-yl)amide
78) 2-Hydroxy-2-(2-methoxybenzyl)-4-methyl-4-phenylpentanoic acid (1-oxo-1,3-dihydroisobenzofuran-5-yl)amide
79) 2-Hydroxy-2-(2-hydroxybenzyl)-4-methyl-4-phenylpentanoic acid (1-oxo-1,3-dihydroisobenzofuran-5-yl)amide
80) 2-Hydroxy-2-[2-(4-hydroxyphenyl)ethyl]4-methyl-4-phenylpentanoic acid (1-oxo-1,3-dihydroisobenzofuran-5-yl)amide
81) 5-[2-Benzyl-4-(5-fluoro-2-methoxyphenyl)-2-hydroxy-4-methylpentylamino]-3H-isobenzofuran-1-one
82) 2-Benzyl-4-(5-fluoro-2-methoxyphenyl)-2-hydroxy-4-methylpentanoic acid (4-cyano-3-trifluoromethylphenyl)amide
83) 2-Benzyl-4-(5-fluoro-2-hydroxyphenyl)-2-hydroxy-4-methylpentanoic acid (4-cyano-3-trifluoromethylphenyl)amide
84) 4-(5-Fluoro-2-methoxyphenyl)-2-hydroxy-4-methyl-2-(1-phenylvinyl)pentanoic acid (1-oxo-1,3-dihydroisobenzofuran-5-yl)amide
85) 2-Hydroxy-4-methyl-4-phenyl-2-pyridin-2-ylmethylpentanoic acid (1-oxo-1,3-dihydroisobenzofuran-5-yl)amide
86) 4-(5-Fluoro-2-methoxyphenyl)-2-hydroxy-4-methyl-2-(1-phenylethyl)pentanoic acid (1-oxo-1,3-dihydroisobenzofuran-5-yl)amide
87) 4-(5-Fluoro-2-hydroxyphenyl)-2-hydroxy-4-methyl-2-(1-phenylethyl)pentanoic acid (1-oxo-1,3-dihydroisobenzofuran-5-yl)amide
88) 2-Benzyl-2-hydroxy-4-methyl-4-phenylpentanoic acid (4-methyl-1-oxo-1H-benzo[d][1,2]oxazin-6-yl)amide
89) 2-Cyclohexylmethyl-2-hydroxy-4-methyl-4-phenylpentanoic acid (4-methyl-1-oxo-1H-benzo[d][1,2]oxazin-6-yl)amide
90) 2-Cyclohexylmethyl-4-(5-fluoro-2-methoxyphenyl)-2-hydroxy-4-methylpentanoic acid (4-cyano-3-trifluoromethylphenyl)amide
91) 2-Cyclopentyl-4-(5-fluoro-2-methoxyphenyl)-2-hydroxy-4-methylpentanoic acid (1-oxo-1,3-dihydroisobenzofuran-5-yl)amide
92) 2-Cyclopentyl-4-(5-fluoro-2-hydroxyphenyl)-2-hydroxy-4-methylpentanoic acid (1-oxo-1,3-dihydroisobenzofuran-5-yl)amide
93) 2-Cyclopentylmethyl-4-(5-fluoro-2-hydroxyphenyl)-2-hydroxy-4-methylpentanoic acid (1-oxo-1,3-dihydroisobenzofuran-5-yl)amide
94) 2-Benzyl-2-hydroxy-N-(1-oxo-1,3-dihydroisobenzofuran-5-yl)-4-phenyl-butyramide
95) 2-Benzyl-2-hydroxy-4-methyl-4-phenylpentanoic acid (1-oxo-1,3-dihydroisobenzofuran-5-yl)amide;
96) 2-Hydroxy-4-methyl-2-phenethyl-4-phenylpentanoic acid (1-oxo-1,3-dihydroisobenzofuran-5-yl)amide;
97) 2-Hydroxy-2-(3-methoxybenzyl)-4-methyl-4-phenylpentanoic acid (1-oxo-1,3-dihydroisobenzofuran-5-yl)amide;
98) 2-Hydroxy-2-[2-(4-methoxyphenyl)ethyl]4-methyl-4-phenylpentanoic acid (1-oxo-1,3-dihydroisobenzofuran-5-yl)amide;
99) 2-Cyclohexylmethyl-2-hydroxy-4-methyl-4-phenylpentanoic acid (1-oxo-1,3-dihydroisobenzofuran-5-yl)amide;
100) 2-(4-tert-Butylbenzyl)-2-hydroxy-4-methyl-4-phenylpentanoic acid (1-oxo-1,3-dihydroisobenzofuran-5-yl)amide;
101) 2-Hydroxy-2-(3-hydroxybenzyl)-4-methyl-4-phenylpentanoic acid (1-oxo-1,3-dihydroisobenzofuran-5-yl)amide;
102) 2-Hydroxy-4-methyl-2-(2-methyl-2-phenylpropyl)-4-phenylpentanoic acid (1-oxo-1,3-dihydroisobenzofuran-5-yl)amide;
103) 2-Benzyl-4-(5-fluoro-2-methoxyphenyl)-2-hydroxy-4-methylpentanoic acid (1-oxo-1,3-dihydroisobenzofuran-5-yl)amide;
104) 2-Cyclohexylmethyl-4-(5-fluoro-2-methoxyphenyl)-2-hydroxy-4-methylpentanoic acid (1-oxo-1,3-dihydroisobenzofuran-5-yl)amide;
105) 2-Benzyl-4-(5-fluoro-2-hydroxyphenyl)-2-hydroxy-4-methylpentanoic acid (1-oxo-1,3-dihydroisobenzofuran-5-yl)amide;
106) 2-Cyclohexylmethyl-4-(5-fluoro-2-hydroxyphenyl)-2-hydroxy-4-methylpentanoic acid (1-oxo-1,3-dihydroisobenzofuran-5-yl)amide;
107) 4-(5-Fluoro-2-hydroxyphenyl)-2-hydroxy-4-methyl-2-(2-methyl-2-phenylpropyl)pentanoic acid (1-oxo-1,3-dihydroisobenzofuran-5-yl)amide;
108) 2-(2-Chloro-6-fluorobenzyl)-4-(5-fluoro-2-methoxyphenyl)-2-hydroxy-4-methylpentanoic acid (1-oxo-1,3-dihydroisobenzofuran-5-yl)amide;
109) 2-(3-Fluorobenzyl)-4-(5-fluoro-2-methoxyphenyl)-2-hydroxy-4-methylpentanoic acid (1-oxo-1,3-dihydroisobenzofuran-5-yl)amide;
110) 2-(2-Fluorobenzyl)-4-(5-fluoro-2-methoxyphenyl)-2-hydroxy-4-methylpentanoic acid (1-oxo-1,3-dihydroisobenzofuran-5-yl)amide;
111) 2-(3,4-Difluorobenzyl)-4-(5-fluoro-2-methoxyphenyl)-2-hydroxy-4-methylpentanoic acid (1-oxo-1,3-dihydroisobenzofuran-5-yl)amide;
112) 2-(2-Chloro-6-fluorobenzyl)-4-(5-fluoro-2-hydroxyphenyl)-2-hydroxy-4-methylpentanoic acid (1-oxo-1,3-dihydroisobenzofuran-5-yl)amide;
113) 2-(3-Fluorobenzyl)-4-(5-fluoro-2-hydroxyphenyl)-2-hydroxy-4-methylpentanoic acid (1-oxo-1,3-dihydroisobenzofuran-5-yl)amide;
114) 2-(2-Fluorobenzyl)-4-(5-fluoro-2-hydroxyphenyl)-2-hydroxy-4-methylpentanoic acid (1-oxo-1,3-dihydroisobenzofuran-5-yl)amide;
115) 2-(3,4-Difluorobenzyl)-4-(5-fluoro-2-hydroxyphenyl)-2-hydroxy-4-methylpentanoic acid (1-oxo-1,3-dihydroisobenzofuran-5-yl)amide;
116) 2-(4-Fluorobenzyl)-4-(5-fluoro-2-methoxyphenyl)-2-hydroxy-4-methylpentanoic acid (1-oxo-1,3-dihydroisobenzofuran-5-yl)amide;
117) 2-(4-Fluorobenzyl)-4-(5-fluoro-2-hydroxyphenyl)-2-hydroxy-4-methylpentanoic acid (1-oxo-1,3-dihydroisobenzofuran-5-yl)amide;
118) 4-(5-Fluoro-2-hydroxyphenyl)-2-hydroxy-4-methyl-2-(3-methylbenzyl)pentanoic acid (1-oxo-1,3-dihydroisobenzofuran-5-yl)amide;
119) 2-Benzyl-4-(5-fluoro-2-methoxyphenyl)-2-hydroxy-4-methylpentanoic acid (4-methyl-1-oxo-1H-benzo[d][1,2]oxazin-6-yl)amide;
120) 2-Benzyl-4-(5-fluoro-2-hydroxyphenyl)-2-hydroxy-4-methylpentanoic acid (4-methyl-1-oxo-1H-benzo[d][1,2]oxazin-6-yl)amide;
121) 2-Cyclohexylmethyl-4-(5-fluoro-2-methoxyphenyl)-2-hydroxy-4-methylpentanoic acid (4-methyl-1-oxo-1H-benzo[d][1,2]oxazin-6-yl)amide;
122) 2-Benzyl-4-(5-fluoro-2-methoxyphenyl)-2-hydroxy-4-methylpentanoic acid (3,5-dichlorophenyl)amide;
123) 4-(5-Fluoro-2-methoxyphenyl)-2-hydroxy-4-methyl-2-(2-methylbenzyl)pentanoic acid (1-oxo-1,3-dihydroisobenzofuran-5-yl)amide;
124) 2-(2,5-Difluorobenzyl)-4-(5-fluoro-2-methoxyphenyl)-2-hydroxy-4-methylpentanoic acid (1-oxo-1,3-dihydroisobenzofuran-5-yl)amide;
125) 2-(2,5-Difluorobenzyl)-4-(5-fluoro-2-hydroxyphenyl)-2-hydroxy-4-methylpentanoic acid (1-oxo-1,3-dihydroisobenzofan-5-yl)amide;
126) 4-(5-Fluoro-2-hydroxyphenyl)-2-hydroxy-4-methyl-2-(2-methylbenzyl)pentanoic acid (1-oxo-1,3-dihydroisobenzofuran-5-yl)amide;
127) 2-Cyclohexylmethyl-4-(5-fluoro-2-methoxyphenyl)-2-hydroxy-4-methylpentanoic acid (3,5-dichlorophenyl)amide;
128) 2-Cyclohexylmethyl-4-(5-fluoro-2-hydroxyphenyl)-2-hydroxy-4-methylpentanoic acid (4-methyl-1-oxo-1H-benzo[d][1,2]oxazin-6-yl)amide;
129) 2-Cyclohexylmethyl-4-(5-fluoro-2-hydroxyphenyl)-2-hydroxy-4-methylpentanoic acid (3,5-dichlorophenyl)amide;
130) 2-(3-Chlorobenzyl-4-(5-fluoro-2-hydroxyphenyl)-2-hydroxy-4-methylpentanoic acid (1-oxo-1,3-dihydroisobenzofuran-5-yl)amide;
131) 4-(5-Fluoro-2-methoxyphenyl)-2-hydroxy-2-(2-methoxybenzyl)-4-methylpentanoic acid (1-oxo-1,3-dihydroisobenzofuran-5-yl)amide;
132) 4-(5-Fluoro-2-methoxyphenyl)-2-hydroxy-4-methyl-2-phenethylpentanoic acid (1-oxo-1,3-dihydroisobenzofuran-5-yl)amide;
133) 2-(2-Chlorobenzyl)-4-(5-fluoro-2-methoxyphenyl)-2-hydroxy-4-methylpentanoic acid (1-oxo-1,3-dihydroisobenzofuran-5-yl)amide;
134) 4-(5-Fluoro-2-hydroxyphenyl)-2-hydroxy-4-methyl-2-phenethylpentanoic acid (1-oxo-1,3-dihydroisobenzofuran-5-yl)amide;
135) 4-(5-Fluoro-2-hydroxyphenyl)-2-hydroxy-2-[2-(4-hydroxyphenyl)ethyl]-4-methylpentanoic acid (1-oxo-1,3-dihydroisobenzofuran-5-yl)amide;
136) 2-(2-Chlorobenzyl)-4-(5-fluoro-2-hydroxyphenyl)-2-hydroxy-4-methylpentanoic acid (1-oxo-1,3-dihydroisobenzofuran-5-yl)amide;
137) 4-(5-Fluoro-2-hydroxyphenyl)-2-hydroxy-2-(2-hydroxybenzyl)-4-methylpentanoic acid (1-oxo-1,3-dihydroisobenzofuran-5-yl)amide;
138) 2-(2-Bromobenzyl)-4-(5-fluoro-2-methoxyphenyl)-2-hydroxy-4-methylpentanoic acid (1-oxo-1,3-dihydroisobenzofuran-5-yl)amide;
139) 2-(2-Bromobenzyl)-4-(5-fluoro-2-hydroxyphenyl)-2-hydroxy-4-methylpentanoic acid (1-oxo-1,3-dihydroisobenzofuran-5-yl)amide;
140) 2-Cyclohexylmethyl-4-(5-fluoro-2-methoxyphenyl)-2-hydroxy-4-methylpentanoic acid (3,5-bistrifluoromethylphenyl)amide;
141) 2-Hydroxy-2-(2-methoxybenzyl)-4-methyl-4-phenylpentanoic acid (1-oxo-1,3-dihydroisobenzofuran-5-yl)amide;
142) 2-Hydroxy-2-(2-hydroxybenzyl)-4-methyl-4-phenylpentanoic acid (1-oxo-1,3-dihydroisobenzofuran-5-yl)amide;
143) 5-[2-Benzyl-4-(5-fluoro-2-methoxyphenyl)-2-hydroxy-4-methylpentylamino]-3H-isobenzofuran-1-one;
144) 2-Benzyl-4-(5-fluoro-2-methoxyphenyl)-2-hydroxy-4-methylpentanoic acid (4-cyano-3-trifluoromethylphenyl)amide;
145) 2-Benzyl-4-(5-fluoro-2-hydroxyphenyl)-2-hydroxy-4-methylpentanoic acid (4-cyano-3-trifluoromethylphenyl)amide;
146) 4-(5-Fluoro-2-methoxyphenyl)-2-hydroxy-4-methyl-2-(1-phenylvinyl)pentanoic acid (1-oxo-1,3-dihydroisobenzofuran-5-yl)amide;
147) 4-(5-Fluoro-2-hydroxyphenyl)-2-hydroxy-4-methyl-2-(1-phenylethyl)pentanoic acid (1-oxo-1,3-dihydroisobenzofuran-5-yl)amide;
148) 2-Benzyl-2-hydroxy-4-methyl-4-phenylpentanoic acid (4-methyl-1-oxo-1H-benzo[d][1,2]oxazin-6-yl)amide;
149) 2-Cyclohexylmethyl-2-hydroxy-4-methyl-4-phenylpentanoic acid (4-methyl-1-oxo-1H-benzo[d][1,2]oxazin-6-yl)amide;
150) 2-Cyclohexylmethyl-4-(5-fluoro-2-methoxyphenyl)-2-hydroxy-4-methylpentanoic acid (4-cyano-3-trifluoromethylphenyl)amide;
151) 2-Cyclopentyl-4-(5-fluoro-2-methoxyphenyl)-2-hydroxy-4-methylpentanoic acid (1-oxo-1,3-dihydroisobenzofuran-5-yl)amide;
152) 2-Cyclopentyl-4-(5-fluoro-2-hydroxyphenyl)-2-hydroxy-4-methylpentanoic acid (1-oxo-1,3-dihydroisobenzofuran-5-yl)amide; and
153) 2-Cyclopentylmethyl-4-(5-fluoro-2-hydroxyphenyl)-2-hydroxy-4-methylpentanoic acid (1-oxo-1,3-dihydroisobenzofuran-5-yl)amide,
154) 2-Benzyl-2-hydroxy-4-methyl-4-phenylpentanoic acid (1-oxo-1,3-dihydroisobenzofuran-5-yl)amide
155) 2-Cyclohexylmethyl-2-hydroxy-4-methyl-4-phenylpentanoic acid (1-oxo-1,3-dihydroisobenzofuran-5-yl)amide;
156) 2-Hydroxy-4-methyl-2-(2-methyl-2-phenylpropyl)-4-phenylpentanoic acid (1-oxo-1,3-dihydroisobenzofuran-5-yl)amide;
157) 2-Benzyl-4-(5-fluoro-2-methoxyphenyl)-2-hydroxy-4-methylpentanoic acid (1-oxo-1,3-dihydroisobenzofuran-5-yl)amide;
158) 2-Cyclohexylmethyl-4-(5-fluoro-2-methoxyphenyl)-2-hydroxy-4-methylpentanoic acid (1-oxo-1,3-dihydroisobenzofuran-5-yl)amide;
159) 2-Cyclohexylmethyl-4-(5-fluoro-2-hydroxyphenyl)-2-hydroxy-4-methylpentanoic acid (1-oxo-1,3-dihydroisobenzofuran-5-yl)amide;
160) 2-(3-Fluorobenzyl)-4-(5-fluoro-2-methoxyphenyl)-2-hydroxy-4-methylpentanoic acid (1-oxo-1,3-dihydroisobenzofuran-5-yl)amide;
161) 2-(2-Fluorobenzyl)-4-(5-fluoro-2-methoxyphenyl)-2-hydroxy-4-methylpentanoic acid (1-oxo-1,3-dihydroisobenzofuran-5-yl)amide;
162) 2-(2-Fluorobenzyl)-4-(5-fluoro-2-hydroxyphenyl)-2-hydroxy-4-methylpentanoic acid (1-oxo-1,3-dihydroisobenzofuran-5-yl)amide;
163) 2-(3,4-Difluorobenzyl)-4-(5-fluoro-2-hydroxyphenyl)-2-hydroxy-4-methylpentanoic acid (1-oxo-1,3-dihydroisobenzofuran-5-yl)amide;
164) 2-(4-Fluorobenzyl)-4-(5-fluoro-2-hydroxyphenyl)-2-hydroxy-4-methylpentanoic acid (1-oxo-1,3-dihydroisobenzofuran-5-yl)amide;
165) 2-Benzyl-4-(5-fluoro-2-methoxyphenyl)-2-hydroxy-4-methylpentanoic acid (4-methyl-1-oxo-1H-benzo[d][1,2]oxazin-6-yl)amide;
166) 2-Benzyl-4-(5-fluoro-2-hydroxyphenyl)-2-hydroxy-4-methylpentanoic acid (4-methyl-1-oxo-1H-benzo[d][1,2]oxazin-6-yl)amide;
167) 2-Cyclohexylmethyl-4-(5-fluoro-2-methoxyphenyl)-2-hydroxy-4-methylpentanoic acid (4-methyl-1-oxo-1H-benzo[d][1,2]oxazin-6-yl)amide;
168) 4-(5-Fluoro-2-methoxyphenyl)-2-hydroxy-4-methyl-2-(2-methylbenzyl)pentanoic acid (1-oxo-1,3-dihydroisobenzofuran-5-yl)amide;
169) 4-(5-Fluoro-2-hydroxyphenyl)-2-hydroxy-4-methyl-2-(2-methylbenzyl)pentanoic acid (1-oxo-1,3-dihydroisobenzofuran-5-yl)amide;
170) 2-Cyclohexylmethyl-4-(5-fluoro-2-hydroxyphenyl)-2-hydroxy-4-methylpentanoic acid (4-methyl-1-oxo-1H-benzo[d][1,2]oxazin-6-yl)amide;
171) 2-(2-Chlorobenzyl)-4-(5-fluoro-2-methoxyphenyl)-2-hydroxy-4-methylpentanoic acid (1-oxo-1,3-dihydroisobenzofuran-5-yl)amide;
172) 4-(5-Fluoro-2-hydroxyphenyl)-2-hydroxy-4-methyl-2-phenethylpentanoic acid (1-oxo-1,3-dihydroisobenzofuran-5-yl)amide;
173) 2-(2-Chlorobenzyl)-4-(5-fluoro-2-hydroxyphenyl)-2-hydroxy-4-methylpentanoic acid (1-oxo-1,3-dihydroisobenzofuran-5-yl)amide;
174) 4-(5-Fluoro-2-hydroxyphenyl)-2-hydroxy-2-(2-hydroxybenzyl)-4-methylpentanoic acid (1-oxo-1,3-dihydroisobenzofuran-5-yl)amide;
175) 2-(2-Bromobenzyl)-4-(5-fluoro-2-methoxyphenyl)-2-hydroxy-4-methylpentanoic acid (1-oxo-1,3-dihydroisobenzofuran-5-yl)amide;
176) 2-(2-Bromobenzyl)-4-(5-fluoro-2-hydroxyphenyl)-2-hydroxy-4-methylpentanoic acid (1-oxo-1,3-dihydroisobenzofuran-5-yl)amide;
177) 5-[2-Benzyl-4-(5-fluoro-2-methoxyphenyl)-2-hydroxy-4-methylpentylamino]-3H-isobenzofuran-1-one;
178) 4-(5-Fluoro-2-hydroxyphenyl)-2-hydroxy-4-methyl-2-(1-phenylethyl)pentanoic acid (1-oxo-1,3-dihydroisobenzofuran-5-yl)amide;
179) 2-Benzyl-2-hydroxy-4-methyl-4-phenylpentanoic acid (4-methyl-1-oxo-1H-benzo[d][1,2]oxazin-6-yl)amide;
180) 2-Cyclohexylmethyl-2-hydroxy-4-methyl-4-phenylpentanoic acid (4-methyl-1-oxo-1H-benzo[d][1,2]oxazin-6-yl)amide;
181) 2-Cyclopentyl-4-(5-fluoro-2-hydroxyphenyl)-2-hydroxy-4-methylpentanoic acid (1-oxo-1,3-dihydroisobenzofuran-5-yl)amide; and
182) 2-Cyclopentylmethyl-4-(5-fluoro-2-hydroxyphenyl)-2-hydroxy-4-methylpentanoic acid (1-oxo-1,3-dihydroisobenzofuran-5-yl)amide.
   Biological Characterization of the Compounds According to the Invention

Progesterone receptor modulators can be identified with the aid of simple methods, test programmes known to the skilled person. It is possible for this purpose for example to incubate a compound to be tested together with a progestogen in a test system for progesterone receptors and to check whether the effect mediated by progesterone is altered in the presence of the modulator in this test system.

The substances according to the invention of the general formula I were tested in the following models:

Progesterone Receptor-Binding Assay

Measurement of the Receptor Binding Affinity:

The receptor binding affinity was determined by competitive binding of a specifically binding ³H-labelled hormone (tracer) and of the compound to be tested on receptors in the cytosol from animal target organs. The aim in this case was receptor saturation and reaction equilibrium.

The tracer and increasing concentrations of the compound to be tested (competitor) were coincubated at 0-4° C. for 18 h with the receptor-containing cytosol fraction. After removal of unbound tracer with carbon-dextran suspension, the receptor-bound tracer content was measured for each concentration, and the IC₅₀ was determined from the concentration series. The relative molar binding affinity (RBA) was calculated as ratio of the IC₅₀ values for reference substance and compound to be tested (×100%) (RBA of the reference substance=100%).

The following incubation conditions were chosen for the receptor types:

Progesterone Receptor:

Uterus cytosol of the estradiol-primed rabbit, homogenized in TED buffer (20 mMTris/HCl, pH 7.4; 1 mM ethylenediaminetetraacetate, 2 mM dithiothreitol) with 250 mM sucrose; stored at −30° C. Tracer: ³H-ORG 2058, 5 nM; reference substance: progesterone.

Glucocorticoid Receptor:

Thymus cytosol from the adrenalectomized rat, thymi stored at −30° C.; buffer: TED.

Tracer: ³H-dexamethasone, 20 nM; reference substance: dexamethasone.

The relative receptor binding affinities (RBA values) for the compounds according to the invention of the general formula (I) on the progesterone receptor are between 3 and 100% relative to progesterone.

The compounds according to the invention accordingly have a high affinity for the progesterone receptor.

Antagonism at the PR-B Progesterone Receptor

The transactivation assay is carried out as described in WO 02/054064.

Agonism at the PR-B Progesterone Receptor

The transactivation assay is carried out as described in Fuhrmann et al. (Fuhrmann U., Hess-Stump H., Cleve A., Neef G., Schwede W., Hoffmann J., Fritzemeier K.-H., Chwalisz K., Journal of Medicinal Chem, 43, 26, 2000, 5010-5016).

Antagonistiic activity

| No. | IC₅₀ [nM] | Efficacy [%] |
|---|---|---|
| 88 | 0.3 | 100 |

Dosage

The progesterone receptor modulators can be administered orally for the use according to the invention.

Satisfactory results are generally to be expected in the treatment of the indications mentioned hereinbefore when the daily doses cover a range from 1 μg to 500 mg of the compound according to the invention.

Suitable dosages of the compounds according to the invention in humans for the treatment of endometriosis, of leiomyomas of the uterus and dysfunctional bleeding and for use in fertility control and for hormone replacement therapy are from 50 μg to 500 mg per day, depending on the age and constitution of the patient, it being possible to administer the necessary daily dose by single or multiple administration.

The dosage range for the compounds according to the invention for the treatment of breast carcinomas is 10 mg to 1000 mg per day.

Suitable for oral administration are in particular tablets, film-coated tablets, other coated tablets, capsules, pills, powders, granules, pastilles, suspensions, emulsions or solutions.

Appropriate tablets can be obtained for example by mixing the active ingredient with known excipients, for example inert diluents such as dextrose, sugar, sorbitol, mannitol, polyvinylpyrrolidone, disintegrants such as corn starch or alginic acid, binders such as starch or gelatin, lubricants such as magnesium stearate or talc and/or means to achieve a depot effect such as carboxypolymethylene, carboxymethylcellulose, cellulose acetate phthalate or polyvinyl acetate. The tablets may also consist of a plurality of layers.

Correspondingly coated tablets can be produced by coating cores produced in analogy to the tablets with compositions normally used in tablet coatings, for example polyvinylpyrrolidone or shellac, gum arabic, talc, titanium oxide or sugar. It is moreover possible for the tablet coating to consists of a plurality of layers, it being possible to use the excipients mentioned above for tablets.

Capsules containing compounds of the general formula I can be produced for example by mixing the compound(s) of the general formula I with an inert carrier such as lactose or sorbitol, and encapsulating in gelatin capsules.

The compounds according to the invention of the general formula (I) or their pharmaceutically acceptable salts can be used, because of their antagonistic or partial agonistic activity, for producing a medicament, in particular for the treatment and prophylaxis of gynaecological disorders such as endometriosis, leiomyomas of the uterus, dysfunctional bleeding and dysmenorrhoea. They can furthermore be employed to counteract hormonal irregularities, for inducing menstruation and alone or in combination with prostaglandins and/or oxytocin to induce labour.

The compounds according to the invention of the general formula (I) or their pharmaceutically acceptable salts are furthermore suitable for producing products for female contraception (see also WO 93/23020, WO 93/21927).

The compounds according to the invention or their pharmaceutically acceptable salts can additionally be employed alone or in combination with a selective estrogen receptor modulator (SERM) for female hormone replacement therapy.

In addition, the said compounds have an antiproliferative effect in hormone-dependent tumours. They are therefore suitable for the therapy of hormone-dependent carcinomas such as, for example, for breast, prostate and endometrial carcinomas.

The compounds according to the invention or their pharmaceutically acceptable salts can be employed for the treatment of hormone-dependent carcinomas both in first-line therapy and in second-line therapy, especially after tamoxifen failure.

The compounds according to the invention, having antagonistic or partial agonistic activity, of the general formula (I) or their pharmaceutically acceptable salts can also be used in combination with compounds having antiestrogenic activity (estrogen receptor antagonists or aromatase inhibitors) or selective estrogen receptor modulators (SERM) for producing pharmaceutical products for the treatment of hormone-dependent tumours. The compounds according to the invention can likewise be used in combination with SERMs or an antiestrogen (estrogen receptor antagonist or aromatase inhibitor) for the treatment of endometriosis or of leiomyomas of the uterus. In the treatment of hormone-dependent tumours the progesterone receptor modulator and the antiestrogen (estrogen receptor antagonists or aromatase inhibitors) or the SERM can be provided for simultaneous or else for sequential administration. In the sequential administration, preferably the antiestrogen (estrogen receptor antagonists or aromatase inhibitors) or SERM is administered first and subsequently the progesterone receptor antagonist or the progesterone receptor modulator is administered.

Suitable for combination with the non-steroidal progesterone receptor modulators according to the invention in this connection are for example the following antiestrogens (estrogen receptor antagonists or aromatase inhibitors) or SERMs: tamoxifen, 5-(4-{5-[(RS)-(4,4,5,5,5-pentafluoropentyl)sulphynyl]pentyloxy}phenyl)-6-phenyl-8,9-dihydro-7H-benzocyclohepten-2-ol (WO 00/03979), ICI 182 780 (7alpha-[9-(4,4,5,5-pentafluoropentylsulphynyl)nonyl]estra-1,3,5(10)-triene-3,17beta-diol), 11beta-fluoro-7alpha-[5-(methyl{3-[(4,4,5,5,5-pentafluoropentyl)sulphanyl]propyl}amino)pentyl]-estra-1,3,5(10)-triene-3,17beta-diol (WO98/07740), 11beta-fluoro-7alpha-{5-[methyl(7,7,8,8,9,9,10,10,10-nonafluorodecyl)amino]pentyl}estra-1,3,5(10)-triene-3,17-beta-diol (WO 99/33855), 11beta-fluoro-17alpha-methyl-7alpha-{5-[methyl(8,8,9,9,9-pentafluorononyl)amino]pentyl}estra-1,3,5(10)-triene-3,17beta-diol (WO 03/045972), clomifen, raloxifen, and further compounds having antiestrogenic activity, and aromatase inhibitors such as, for example, fadrozole, formestane, letrozole, anastrozole or atamestane.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosures of all applications, patents and publications, cited herein and of corresponding German application No. 102005030293.9, filed Jun. 24, 2005, and U.S. Provisional Application Ser. No. 60/693,414, filed Jun. 24, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Use of non-steroidal compounds of the general formula I 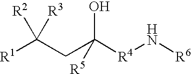
in which
R¹ is an aryl or heteroaryl group which is unsubstituted or optionally substituted by up to 3 radicals, where the substituents each independently of one another have the following meaning:
C₁-C₅-alkyl, C₂-C₅-alkenyl, C₂-C₅-alkynyl, C₃-C₈-cycloalkyl, aryl, C₁-C₅-alkoxy, aryloxy, C₁-C₅-alkanoyl, aroyl, C₁-C₅-alkoxycarbonyl, C₁-C₅-alkanoyloxy, aminocarbonyloxy, C₁-C₅-alkylaminocarbonyloxy, C₁-C₅-dialkylaminocarbonyloxy, aminocarbonyl, C₁-C₅-alkylaminocarbonyl, C₁-C₅-dialkylaminocarbonyl, C₁-C₅-alkanoylamino, C₁-C₅-alkoxycarbonylamino, C₁-C₅-alkylsulphonylamino, C₁-C₅-alkylaminosulphonyl, C₁-C₅-dialkylaminosulphonyl, halogen, hydroxy, carboxy, cyano, trifluoromethyl, nitro, amino, with a mono- or disubstituted nitrogen atom, where the substituents on the nitrogen are each independently of one another
C₁-C₅-alkyl or aryl or ureido, in which each nitrogen is substituted independently of the other by C₁-C₅-alkyl or C₁-C₅-alkylthio, where the sulphur may optionally be oxidized to a sulphoxide or sulphone, or in which each substituent may in turn be substituted independently of one another in each case by 1-3 radicals of the following meaning: methyl, halogen, hydroxy, oxo, cyano, trifluoromethyl and amino,
R² and R³ are each independently of one another hydrogen, C₁-C₅-alkyl, or
R² and R³ afford together with the C atom of the chain a ring having a total of 3-8 carbon atoms
R⁴ is CH₂ or C═O;
R⁵ is a carbocyclic, heterocyclic, aromatic or heteroaromatic ring which is attached directly or via a C₁-C₈-alkyl or a C₂-C₈-alkenyl and is unsubstituted or optionally substituted by 1-3 radicals, where each substituent in turn may be substituted independently of one another in each case by 1-3 radicals of the following meaning:
C₁-C₅-alkyl, C₂-C₅-alkenyl, C₂-C₅-alkynyl, C₃-C₈-cycloalkyl, phenyl, C₁-C₅-alkoxy, phenoxy, C₁-C₅-alkanoyl, aroyl, C₁-C₅-alkoxycarbonyl, C₁-C₅-alkanoyloxy, aminocarbonyloxy, C₁-C₅-alkylaminocarbonyloxy, C₁-C₅-dialkylaminocarbonyloxy, aminocarbonyl, C₁-C₅-alkylaminocarbonyl, C₁-C₅-dialkylaminocarbonyl, C₁-C₅-alkanoylamino, C₁-C₅-alkoxycarbonylamino, C₁-C₅-alkylsulphonylamino, C₁-C₅-alkylaminosulphonyl, C₁-C₅-dialkylaminosulphonyl, halogen, hydroxy, carboxy, cyano, trifluoromethyl, nitro, amino, with a mono- or disubstituted nitrogen atom, where the substituents on the nitrogen are each independently of one another C₁-C₅-alkyl or aryl or ureido, in which each nitrogen is substituted independently of the other by C₁-C₅-alkyl or C₁-C₅-alkylthio, where the sulphur may optionally be oxidized to a sulphoxide or sulphone, and
R⁶ is an aromatic system which is unsubstituted or optionally substituted by 1-3 radicals, or is a group A or B: 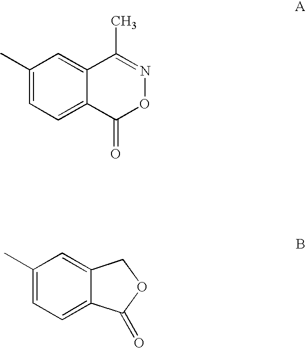
in which each substituent on R⁶ has independently of one another the following meaning:
C₁-C₅-alkyl, C₂-C₅-alkenyl, C₂-C₅-alkynyl, C₃-C₈-cycloalkyl, C₁-C₅-alkoxy, C₁-C₅-alkanoyl, C₁-C₅-alkoxycarbonyl, C₁-C₅-alkanoyloxy, aminocarbonyloxy, C₁-C₅-alkylaminocarbonyloxy, C₁-C₅-dialkylaminocarbonyloxy, aminocarbonyl, C₁-C₅-alkylaminocarbonyl, C₁-C₅-dialkylaminocarbonyl, C₁-C₅-alkanoylamino, C₁-C₅-alkoxycarbonylamino, C₁-C₅-alkylsulphonylamino, C₁-C₅-alkylaminosulphonyl, C₁-C₅-dialkylaminosulphonyl, halogen, hydroxy, carboxy, cyano, trifluoromethyl, nitro, amino, with a mono- or disubstituted nitrogen atom, where the substituents on the nitrogen are each independently of one another
C₁-C₅-alkyl or aryl or ureido, in which each nitrogen is substituted independently of the other by C₁-C₅-alkyl or C₁-C₅-alkylthio, where the sulphur may optionally be oxidized to a sulphoxide or sulphone,
or their tautomers, prodrugs, solvates, or salts for the therapy and prophylaxis of gynaecological disorders such as endometriosis, leiomyomas of the uterus, dysfunctional bleeding and dysmenorrhoea.

2. Use of compounds of the general formula (I) for the therapy and prophylaxis of hormone-dependent tumours.

3. Use of compounds of the general formula (I) for the therapy and prophylaxis of breast carcinomas.

4. Use of compounds of the general formula (I) for the therapy and prophylaxis of endometrial carcinoma.

5. Use of compounds of the general formula (I) for the therapy and prophylaxis of ovarian carcinomas.

6. Use of compounds of the general formula (I) for the therapy and prophylaxis of prostate carcinomas.

7. Use of compounds of the general formula (I) for female hormone replacement therapy.

8. Use of compounds of the general formula (I) for female fertility control.

9. Use of compounds according to claim 1 in which R¹ is preferably an unsubstituted or substituted phenyl, naphthyl, indanyl, indenyl, chromanyl, dihydrobenzofuranyl, dihydroindolyl, dihydroquinolinyl, dihydroisoquinolinyl, tetrahydroquinolinyl, tetrahydroisoquinolinyl, thienyl, furanyl, pyrrolyl, pyridinyl, pyrimidinyl, pyrazinyl, indolyl, benzofuranyl, or benzothienyl radical.

10. Use of compounds according to claim 9 in which the radicals mentioned for R¹ are in each case substituted independently of one another by methyl, fluorine, chlorine, bromine, hydroxy, oxo, cyano, trifluoromethyl and amino.

11. Use of compounds according to claim 1 in which R² and R³ are independently of one another C₁-C₃-alkyl.

12. Use of compounds according to claim 1 in which R² and R³ form together with the C atom of the chain a ring having a total of 3-8 carbon atoms.

13. Use of compounds according to claim 1 in which R⁴ is CH₂.

14. Use of compounds according to claim 1 in which R⁴ is C═O.

15. Use of compounds according to claim 1 in which R⁵ is substituted or unsubstituted cyclopropyl, cyclobutyl, cyclopentyl, cyclohexyl, cyclopentylmethyl, cyclohexylmethyl, benzyl, cyclopentylethyl, cyclohexylethyl, phenethyl or phenyldifluoromethyl.

16. Use of compounds according to claim 15 in which the radicals mentioned for R⁵ are in each case substituted independently of one another by methyl, methoxy, hydroxy, halogen, cyano or trifluoromethyl.

17. Use of compounds according to claim 1 in which R⁶ is an unsubstituted or substituted phenyl group.

18. Use of compounds according to claim 1 in which R⁶ is a group A or B:

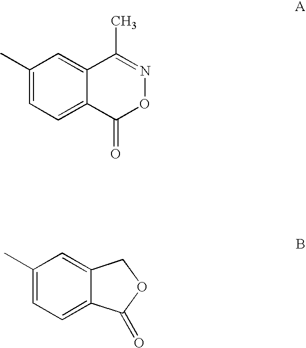

19. Use of compounds according to claim 18 in which the radicals mentioned for R⁶ in each case mean independently of one another methyl, methoxy, halogen, cyano or trifluoromethyl.

20. Use of compounds according to claim 1 in which the halogen atoms are preferably chlorine and/or fluorine.

21. Use of compounds according to claim 1, namely
1) 2-Benzyl-2-hydroxy-4-methyl-4-phenylpentanoic acid (1-oxo-1,3-dihydroisobenzofuran-5-yl)amide
2) 2-Hydroxy-4-methyl-2,4-diphenylpentanoic acid (1-oxo-1,3-dihydroisobenzofuran-5-yl)amide
3) 2-Hydroxy-4-methyl-2-phenethyl-4-phenylpentanoic acid (1-oxo-1,3-dihydroisobenzo-furan-5-yl)amide
4) 2-Hydroxy-2-(3-methoxybenzyl)-4-methyl-4-phenylpentanoic acid (1-oxo-1,3-dihydro-isobenzofuran-5-yl)amide
5) 2-Hydroxy-2-(4-methoxybenzyl)-4-methyl-4-phenylpentanoic acid (1-oxo-1,3-dihydroisobenzofuran-5-yl)amide
6) 2-Hydroxy-2-[2-(4-methoxyphenyl)ethyl]-4-methyl-4-phenylpentanoic acid (1-oxo-1,3-dihydroisobenzofuran-5-yl)amide
7) 2-Cyclohexylmethyl-2-hydroxy-4-methyl-4-phenylpentanoic acid (1-oxo-1,3-dihydroisobenzofuran-5-yl)amide
8) 2-(4-tert-Butylbenzyl)-2-hydroxy-4-methyl-4-phenylpentanoic acid (1-oxo-1,3-dihydroisobenzofuran-5-yl)amide
9) 2-Biphenyl-4-ylmethyl-2-hydroxy-4-methyl-4-phenylpentanoic acid (1-oxo-1,3-dihydroisobenzofuran-5-yl)amide
10) 2-Hydroxy-4-methyl-2-naphthalen-2-ylmethyl-4-phenylpentanoic acid (1-oxo-1,3-dihydroisobenzofuran-5-yl)amide
11) 2-Hydroxy-2-(3-hydroxybenzyl)-4-methyl-4-phenylpentanoic acid (1-oxo-1,3-dihydroisobenzofuran-5-yl)amide
12) 2-Hydroxy-4-methyl-2-(2-methyl-2-phenylpropyl)-4-phenylpentanoic acid (1-oxo-1,3-dihydroisobenzofuran-5-yl)amide
13) 2-Benzyl-4-(5-fluoro-2-methoxyphenyl)-2-hydroxy-4-methylpentanoic acid (1-oxo-1,3-dihydroisobenzofuran-5-yl)amide
14) 2-Cyclohexylmethyl-4-(5-fluoro-2-methoxyphenyl)-2-hydroxy-4-methylpentanoic acid (1-oxo-1,3-dihydroisobenzofuran-5-yl)amide
15) 2-Benzyl-4-(5-fluoro-2-hydroxyphenyl)-2-hydroxy-4-methylpentanoic acid (1-oxo-1,3-dihydroisobenzofuran-5-yl)amide
16) 2-Cyclohexylmethyl-4-(5-fluoro-2-hydroxyphenyl)-2-hydroxy-4-methylpentanoic acid (1-oxo-1,3-dihydroisobenzofuran-5-yl)amide
17) 4-(5-Fluoro-2-hydroxyphenyl)-2-hydroxy-4-methyl-2-(2-methyl-2-phenylpropyl)pentanoic acid (1-oxo-1,3-dihydroisobenzofuran-5-yl)amide
18) 2-(2-Chloro-6-fluorobenzyl)-4-(5-fluoro-2-methoxyphenyl)-2-hydroxy-4-methylpentanoic acid (1-oxo-1,3-dihydroisobenzofuran-5-yl)amide
19) 2-(3-Fluorobenzyl)-4-(5-fluoro-2-methoxyphenyl)-2-hydroxy-4-methylpentanoic acid (1-oxo-1,3-dihydroisobenzofuran-5-yl)amide
20) 2-(2-Fluorobenzyl)-4-(5-fluoro-2-methoxyphenyl)-2-hydroxy-4-methylpentanoic acid (1-oxo-1,3-dihydroisobenzofuran-5-yl)amide
21) 2-(3,4-Difuorobenzyl)-4-(5-fluoro-2-methoxyphenyl)-2-hydroxy-4-methylpentanoic acid (1-oxo-1,3-dihydroisobenzofuran-5-yl)amide
22) 2-(2-Chloro-6-fluorobenzyl)-4-(5-fluoro-2-hydroxyphenyl)-2-hydroxy-4-methylpentanoic acid (1-oxo-1,3-dihydroisobenzofuran-5-yl)amide
23) 2-(3-Fluorobenzyl)-4-(5-fluoro-2-hydroxyphenyl)-2-hydroxy-4-methylpentanoic acid (1-oxo-1,3-dihydroisobenzofuran-5-yl)amide
24) 2-(2-Fluorobenzyl)-4-(5-fluoro-2-hydroxyphenyl)-2-hydroxy-4-methylpentanoic acid (1-oxo-1,3-dihydroisobenzofuran-5-yl)amide
25) 2-(3,4-Difluorobenzyl)-4-(5-fluoro-2-hydroxyphenyl)-2-hydroxy-4-methylpentanoic acid (1-oxo-1,3-dihydroisobenzofuran-5-yl)amide
26) 2-(4-Fluorobenzyl)-4-(5-fluoro-2-methoxyphenyl)-2-hydroxy-4-methylpentanoic acid (1-oxo-1,3-dihydroisobenzofuran-5-yl)amide
27) 4-(5-Fluoro-2-methoxyphenyl)-2-hydroxy-4-methyl-2-(3-methylbenzyl)pentanoic acid (1-oxo-1,3-dihydroisobenzofuran-5-yl)amide
28) 2-(4-Fluorobenzyl)-4-(5-fluoro-2-hydroxyphenyl)-2-hydroxy-4-methylpentanoic acid (1-oxo-1,3-dihydroisobenzofuran-5-yl)amide
29) 4-(5-Fluoro-2-hydroxyphenyl)-2-hydroxy-4-methyl-2-(3-methylbenzyl)pentanoic acid (1-oxo-1,3-dihydroisobenzofuran-5-yl)amide
30) 2-(3,5-Difluorophenyl)-4-(5-fluoro-2-hydroxyphenyl)-2-hydroxy-4-methylpentanoic acid (1-oxo-1,3-dihydroisobenzofuran-5-yl)amide
31) 2-Benzyl-4-(5-fluoro-2-methoxyphenyl)-2-hydroxy-4-methylpentanoic acid (4-methyl-1-oxo-1H-benzo[d][1,2]oxazin-6-yl)amide
32) 2-Benzyl-4-(5-fluoro-2-hydroxyphenyl)-2-hydroxy-4-methylpentanoic acid (4-methyl-1-oxo-1H-benzo[d][1,2]oxazin-6-yl)amide
33) 2-Cyclohexylmethyl-4-(5-fluoro-2-methoxyphenyl)-2-hydroxy-4-methylpentanoic acid (4-methyl-1-oxo-1H-benzo[d][1,2]oxazin-6-yl)amide
34) 2-Benzyl-4-(5-fluoro-2-methoxyphenyl)-2-hydroxy-4-methylpentanoic acid (3,5-dichlorophenyl)amide
35) 4-(5-Fluoro-2-methoxyphenyl)-2-hydroxy-4-methyl-2-(2-methylbenzyl)pentanoic acid (1-oxo-1,3-dihydroisobenzofuran-5-yl)amide
36) 2-(3,5-Dimethylbenzyl)-4-(5-fluoro-2-methoxyphenyl)-2-hydroxy-4-methylpentanoic acid (1-oxo-1,3-dihydroisobenzofuran-5-yl)amide
37) 2-(2,5-Difluorobenzyl)-4-(5-fluoro-2-methoxyphenyl)-2-hydroxy-4-methylpentanoic acid (1-oxo-1,3-dihydroisobenzofuran-5-yl)amide
38) 2-(2,5-Difluorobenzyl)-4-(5-fluoro-2-hydroxyphenyl)-2-hydroxy-4-methylpentanoic acid (1-oxo-1,3-dihydroisobenzofuran-5-yl)amide
39) 4-(5-Fluoro-2-hydroxyphenyl)-2-hydroxy-4-methyl-2-(2-methylbenzyl)pentanoic acid (1-oxo-1,3-dihydroisobenzofuran-5-yl)amide
40) 2-(3,5-Dimethylbenzyl)-4-(5-fluoro-2-hydroxyphenyl)-2-hydroxy-4-methylpentanoic acid (1-oxo-1,3-dihydroisobenzofuran-5-yl)amide
41) 2-Cyclohexylmethyl-4-(5-fluoro-2-methoxyphenyl)-2-hydroxy-4-methylpentanoic acid (3,5-dichlorophenyl)amide
42) 2-Cyclohexylmethyl-4-(5-fluoro-2-hydroxyphenyl)-2-hydroxy-4-methylpentanoic acid (4-methyl-1-oxo-1H-benzo[d][1,2]oxazin-6-yl)amide
43) 2-Benzyl-4-(5-fluoro-2-hydroxyphenyl)-2-hydroxy-4-methylpentanoic acid (3,5-dichlorophenyl)amide
44) 2-Cyclohexylmethyl-4-(5-fluoro-2-hydroxyphenyl)-2-hydroxy-4-methylpentanoic acid (3,5-dichlorophenyl)amide
45) 2-Benzyl-4-(5-fluoro-2-methoxyphenyl)-2-hydroxy-4-methylpentanoic acid phenylamide
46) 2-Cyclohexylmethyl-4-(5-fluoro-2-methoxyphenyl)-2-hydroxy-4-methylpentanoic acid phenylamide
47) 2-(3-Chlorobenzyl)-4-(5-fluoro-2-hydroxyphenyl)-2-hydroxy-4-methylpentanoic acid (1-oxo-1,3-dihydroisobenzofuran-5-yl)amide
48) 4-(5-Fluoro-2-methoxyphenyl)-2-hydroxy-2-[2-(4-methoxyphenyl)ethyl]-4-methylpentanoic acid (1-oxo-1,3-dihydroisobenzofuran-5-yl)amide
49) 4-(5-Fluoro-2-methoxyphenyl)-2-hydroxy-2-(2-methoxybenzyl)-4-methylpentanoic acid (1-oxo-1,3-dihydroisobenzofuran-5-yl)amide
50) 2-Benzyl-4-(5-fluoro-2-hydroxyphenyl)-2-hydroxy-4-methylpentanoic acid phenylamide
51) 2-Cyclohexylmethyl-4-(5-fluoro-2-hydroxyphenyl)-2-hydroxy-4-methylpentanoic acid phenylamide
52) 4-(5-Fluoro-2-methoxyphenyl)-2-hydroxy-4-methyl-2-phenethylpentanoic acid (1-oxo-1,3-dihydroisobenzofuran-5-yl)amide
53) 2-(2-Chlorobenzyl)-4-(5-fluoro-2-methoxyphenyI)-2-hydroxy-4-methylpentanoic acid (1-oxo-1,3-dihydroisobenzofuran-5-yl)amide
54) 4-(5-Fluoro-2-hydroxyphenyl)-2-hydroxy-4-methyl-2-phenethylpentanoic acid (1-oxo-1,3-dihydroisobenzofuran-5-yl)amide
55) 4-(5-Fluoro-2-hydroxyphenyl)-2-hydroxy-2-[2-(4-hydroxyphenyl)ethyl]-4-methylpentanoic acid (1-oxo-1,3-dihydroisobenzofuran-5-yl)amide
56) 2-(2-Chlorobenzyl)-4-(5-fluoro-2-hydroxyphenyl)-2-hydroxy-4-methylpentanoic acid (1-oxo-1,3-dihydroisobenzofuran-5-yl)amide
57) 4-(5-Fluoro-2-hydroxyphenyl)-2-hydroxy-2-(2-hydroxybenzyl)-4-methylpentanoic acid (1-oxo-1,3-dihydroisobenzofuran-5-yl)amide
58) 2-(2-Bromobenzyl)-4-(5-fluoro-2-methoxyphenyl)-2-hydroxy-4-methylpentanoic acid (1-oxo-1,3-dihydroisobenzofuran-5-yl)amide
59) 2-(2-Bromobenzyl)-4-(5-fluoro-2-hydroxyphenyl)-2-hydroxy-4-methylpentanoic acid (1-oxo-1,3-dihydroisobenzofuran-5-yl)amide
60) 2-Benzyl-4-(5-fluoro-2-methoxyphenyl)-2-hydroxy-4-methylpentanoic acid (3,5-dimethylphenyl)amide
61) 2-Cyclohexylmethyl-4-(5-fluoro-2-methoxyphenyl)-2-hydroxy-4-methylpentanoic acid (3,5-dimethylphenyl)amide
62) 2-Benzyl-4-(5-fluoro-2-methoxyphenyl)-2-hydroxy-4-methylpentanoic acid (3,5-bistrifluoromethylphenyl)amide
63) 2-Cyclohexylmethyl-4-(5-fluoro-2-methoxyphenyl)-2-hydroxy-4-methylpentanoic acid (3,5-bistrifluoromethylphenyl)amide
64) 2-Benzyl-4-(5-fluoro-2-methoxyphenyl)-2-hydroxy-4-methylpentanoic acid (3,5-dimethoxyphenyl)amide
65) 2-Cyclohexylmethyl-4-(5-fluoro-2-methoxyphenyl)-2-hydroxy-4-methylpentanoic acid (3,5-dimethoxyphenyl)amide
66) 2-Benzyl-4-(5-fluoro-2-hydroxyphenyl)-2-hydroxy-4-methylpentanoic acid (3,5-dimethylphenyl)amide
67) 2-Cyclohexylmethyl-4-(5-fluoro-2-hydroxyphenyl)-2-hydroxy-4-methylpentanoic acid (3,5-dimethylphenyl)amide
68) 2-Benzyl-4-(5-fluoro-2-hydroxyphenyl)-2-hydroxy-4-methylpentanoic acid (3,5-bistrifluoromethylphenyl)amide
69) 2-Cyclohexylmethyl-4-(5-fluoro-2-hydroxyphenyl)-2-hydroxy-4-methylpentanoic acid (3,5-bistrifluoromethylphenyl)amide
70) 2-Benzyl-4-(5-fluoro-2-hydroxyphenyl)-2-hydroxy-4-methylpentanoic acid (3,5-dihydroxyphenyl)amide
71) 2-Cyclohexylmethyl-4-(5-fluoro-2-hydroxyphenyl)-2-hydroxy-4-methylpentanoic acid (3,5-dihydroxyphenyl)amide
72) 2-(5-Fluoro-2-methoxybenzyl)-2-hydroxy-4-methyl-4-phenylpentanoic acid (1-oxo-1,3-dihydroisobenzofuran-5-yl)amide
73) 2-(5-Fluoro-2-hydroxybenzyl)-2-hydroxy-4-methyl-4-phenylpentanoic acid (1-oxo-1,3-dihydroisobenzofuran-5-yl)amide
74) 2-(5-Fluoro-2-methoxybenzyl)-4-(5-fluoro-2-methoxyphenyl)-2-hydroxy-4-methylpentanoic acid (1-oxo-1,3-dihydroisobenzofuran-5-yl)amide
75) 2-(5-Fluoro-2-hydroxybenzyl)-4-(5-fluoro-2-hydroxyphenyl)-2-hydroxy-4-methylpentanoic acid (1-oxo-1,3-dihydroisobenzofuran-5-yl)amide
76) 2-(3,5-Dimethoxybenzyl)-2-hydroxy-4-methyl-4-phenylpentanoic acid (1-oxo-1,3-dihydroisobenzofuran-5-yl)amide
77) 2-(3,5-Dihydroxybenzyl)-2-hydroxy-4-methyl-4-phenylpentanoic acid (1-oxo-1,3-dihydroisobenzofuran-5-yl)amide
78) 2-Hydroxy-2-(2-methoxybenzyl)-4-methyl-4-phenylpentanoic acid (1-oxo-1,3-dihydroisobenzofuran-5-yl)amide
79) 2-Hydroxy-2-(2-hydroxybenzyl)-4-methyl-4-phenylpentanoic acid (1-oxo-1,3-dihydroisobenzofuran-5-yl)amide
80) 2-Hydroxy-2-[2-(4-hydroxyphenyl)ethyl]-4-methyl-4-phenylpentanoic acid (1-oxo-1,3-dihydroisobenzofuran-5-yl)amide
81) 5-[2-Benzyl-4-(5-fluoro-2-methoxyphenyl)-2-hydroxy-4-methylpentylamino]-3H-isobenzofuran-1-one
82) 2-Benzyl-4-(5-fluoro-2-methoxyphenyl)-2-hydroxy-4-methylpentanoic acid (4-cyano-3-trifluoromethylphenyl)amide
83) 2-Benzyl-4-(5-fluoro-2-hydroxyphenyl)-2-hydroxy-4-methylpentanoic acid (4-cyano-3-trifluoromethylphenyl)amide
84) 4-(5-Fluoro-2-methoxyphenyl)-2-hydroxy-4-methyl-2-(1-phenylvinyl)pentanoic acid (1-oxo-1,3-dihydroisobenzofuran-5-yl)amide
85) 2-Hydroxy-4-methyl-4-phenyl-2-pyridin-2-ylmethylpentanoic acid (1-oxo-1,3-dihydroisobenzofuran-5-yl)amide
86) 4-(5-Fluoro-2-methoxyphenyl)-2-hydroxy-4-methyl-2-(1-phenylethyl)pentanoic acid (1-oxo-1,3-dihydroisobenzofuran-5-yl)amide
87) 4-(5-Fluoro-2-hydroxyphenyl)-2-hydroxy-4-methyl-2-(1-phenylethyl)pentanoic acid (1-oxo-1,3-dihydroisobenzofuran-5-yl)amide
88) 2-Benzyl-2-hydroxy-4-methyl-4-phenylpentanoic acid (4-methyl-1-oxo-1H-benzo[d][1,2]oxazin-6-yl)amide
89) 2-Cyclohexylmethyl-2-hydroxy-4-methyl-4-phenylpentanoic acid (4-methyl-1-oxo-1H-benzo[d][1,2]oxazin-6-yl)amide
90) 2-Cyclohexylmethyl-4-(5-fluoro-2-methoxyphenyl)-2-hydroxy-4-methylpentanoic acid (4-cyano-3-trifluoromethylphenyl)amide
91) 2-Cyclopentyl-4-(5-fluoro-2-methoxyphenyl)-2-hydroxy-4-methylpentanoic acid (1-oxo-1,3-dihydroisobenzofuran-5-yl)amide
92) 2-Cyclopentyl-4-(5-fluoro-2-hydroxyphenyl)-2-hydroxy-4-methylpentanoic acid (1-oxo-1,3-dihydroisobenzofuran-5-yl)amide
93) 2-Cyclopentylmethyl-4-(5-fluoro-2-hydroxyphenyl)-2-hydroxy-4-methylpentanoic acid (1-oxo-1,3-dihydroisobenzofuran-5-yl)amide
94) 2-Benzyl-2-hydroxy-N-(1-oxo-1,3-dihydroisobenzofuran-5-yl)-4-phenyl-butyramide
95) 2-Benzyl-2-hydroxy-4-methyl-4-phenylpentanoic acid (1-oxo-1,3-dihydroisobenzofuran-5-yl)amide;
96) 2-Hydroxy-4-methyl-2-phenethyl-4-phenylpentanoic acid (1-oxo-1,3-dihydroisobenzofuran-5-yl)amide;
97) 2-Hydroxy-2-(3-methoxybenzyl)-4-methyl-4-phenylpentanoic acid (1-oxo-1,3-dihydroisobenzofuran-5-yl)amide;
98) 2-Hydroxy-2-[2-(4-methoxyphenyl)ethyl]-4-methyl-4-phenylpentanoic acid (1-oxo-1,3-dihydroisobenzofuran-5-yl)amide;
99) 2-Cyclohexylmethyl-2-hydroxy-4-methyl-4-phenylpentanoic acid (1-oxo-1,3-dihydroisobenzofuran-5-yl)amide;
100) 2-(4-tert-Butylbenzyl)-2-hydroxy-4-methyl-4-phenylpentanoic acid (1-oxo-1,3-dihydroisobenzofuran-5-yl)amide;
101) 2-Hydroxy-2-(3-hydroxybenzyl)-4-methyl-4-phenylpentanoic acid (1-oxo-1,3-dihydroisobenzofuran-5-yl)amide;
102) 2-Hydroxy-4-methyl-2-(2-methyl-2-phenylpropyl)-4-phenylpentanoic acid (1-oxo-1,3-dihydroisobenzofuran-5-yl)amide;
103) 2-Benzyl-4-(5-fluoro-2-methoxyphenyl)-2-hydroxy-4-methylpentanoic acid (1-oxo-1,3-dihydroisobenzofuran-5-yl)amide;
104) 2-Cyclohexylmethyl-4-(5-fluoro-2-methoxyphenyl)-2-hydroxy-4-methylpentanoic acid (1-oxo-1,3-dihydroisobenzofuran-5-yl)amide;
105) 2-Benzyl-4-(5-fluoro-2-hydroxyphenyl)-2-hydroxy-4-methylpentanoic acid (1-oxo-1,3-dihydroisobenzofuran-5-yl)amide;
106) 2-Cyclohexylmethyl-4-(5-fluoro-2-hydroxyphenyl)-2-hydroxy-4-methylpentanoic acid (1-oxo-1,3-dihydroisobenzofuran-5-yl)amide;
107) 4-(5-Fluoro-2-hydroxyphenyl)-2-hydroxy-4-methyl-2-(2-methyl-2-phenylpropyl)pentanoic acid (1-oxo-1,3-dihydroisobenzofuran-5-yl)amide;
108) 2-(2-Chloro-6-fluorobenzyl)-4-(5-fluoro-2-methoxyphenyl)-2-hydroxy-4-methylpentanoic acid (1-oxo-1,3-dihydroisobenzofuran-5-yl)amide;
109) 2-(3-Fluorobenzyl)-4-(5-fluoro-2-methoxyphenyl)-2-hydroxy-4-methylpentanoic acid (1-oxo-1,3-dihydroisobenzofuran-5-yl)amide;
110) 2-(2-Fluorobenzyl)-4-(5-fluoro-2-methoxyphenyl)-2-hydroxy-4-methylpentanoic acid (1-oxo-1,3-dihydroisobenzofuran-5-yl)amide;
111) 2-(3,4-Difluorobenzyl)-4-(5-fluoro-2-methoxyphenyl)-2-hydroxy-4-methylpentanoic acid (1-oxo-1,3-dihydroisobenzofuran-5-yl)amide;
112) 2-(2-Chloro-6-fluorobenzyl)-4-(5-fluoro-2-hydroxyphenyl)-2-hydroxy-4-methylpentanoic acid (1-oxo-1,3-dihydroisobenzofuran-5-yl)amide;
113) 2-(3-Fluorobenzyl)-4-(5-fluoro-2-hydroxyphenyl)-2-hydroxy-4-methylpentanoic acid (1-oxo-1,3-dihydroisobenzofuran-5-yl)amide;
114) 2-(2-Fluorobenzyl)-4-(5-fluoro-2-hydroxyphenyl)-2-hydroxy-4-methylpentanoic acid (1-oxo-1,3-dihydroisobenzofuran-5-yl)amide;
115) 2-(3,4-Difluorobenzyl)-4-(5-fluoro-2-hydroxyphenyl)-2-hydroxy-4-methylpentanoic acid (1-oxo-1,3-dihydroisobenzofuran-5-yl)amide;
116) 2-(4-Fluorobenzyl)-4-(5-fluoro-2-methoxyphenyl)-2-hydroxy-4-methylpentanoic acid (1-oxo-1,3-dihydroisobenzofuran-5-yl)amide;
117) 2-(4-Fluorobenzyl)-4-(5-fluoro-2-hydroxyphenyl)-2-hydroxy-4-methylpentanoic acid (1-oxo-1,3-dihydroisobenzofuran-5-yl)amide;
118) 4-(5-Fluoro-2-hydroxyphenyl)-2-hydroxy-4-methyl-2-(3-methylbenzyl)pentanoic acid (1-oxo-1,3-dihydroisobenzofuran-5-yl)amide;
119) 2-Benzyl-4-(5-fluoro-2-methoxyphenyl)-2-hydroxy-4-methylpentanoic acid (4-methyl-1-oxo-1H-benzo[d][1,2]oxazin-6-yl)amide;
120) 2-Benzyl-4-(5-fluoro-2-hydroxyphenyl)-2-hydroxy-4-methylpentanoic acid (4-methyl-1-oxo-1H-benzo[d][1,2]oxazin-6-yl)amide;
121) 2-Cyclohexylmethyl-4-(5-fluoro-2-methoxyphenyl)-2-hydroxy-4-methylpentanoic acid (4-methyl-1-oxo-1H-benzo[d][1,2]oxazin-6-yl)amide;
122) 2-Benzyl-4-(5-fluoro-2-methoxyphenyl)-2-hydroxy-4-methylpentanoic acid (3,5-dichlorophenyl)amide;
123) 4-(5-Fluoro-2-methoxyphenyl)-2-hydroxy-4-methyl-2-(2-methylbenzyl)pentanoic acid (1-oxo-1,3-dihydroisobenzofuran-5-yl)amide;
124) 2-(2,5-Difluorobenzyl)-4-(5-fluoro-2-methoxyphenyl)-2-hydroxy-4-methylpentanoic acid (1-oxo-1,3-dihydroisobenzofuran-5-yl)amide;
125) 2-(2,5-Difluorobenzyl)-4-(5-fluoro-2-hydroxyphenyl)-2-hydroxy-4-methylpentanoic acid (1-oxo-1,3-dihydroisobenzofan-5-yl)amide;
126) 4-(5-Fluoro-2-hydroxyphenyl)-2-hydroxy-4-methyl-2-(2-methylbenzyl)pentanoic acid (1-oxo-1,3-dihydroisobenzofuran-5-yl)amide;
127) 2-Cyclohexylmethyl-4-(5-fluoro-2-methoxyphenyl)-2-hydroxy-4-methylpentanoic acid (3,5-dichlorophenyl)amide;
128) 2-Cyclohexylmethyl-4-(5-fluoro-2-hydroxyphenyl)-2-hydroxy-4-methylpentanoic acid (4-methyl-1-oxo-1H-benzo[d][1,2]oxazin-6-yl)amide;
129) 2-Cyclohexylmethyl-4-(5-fluoro-2-hydroxyphenyl)-2-hydroxy-4-methylpentanoic acid (3,5-dichlorophenyl)amide;
130) 2-(3-Chlorobenzyl-4-(5-fluoro-2-hydroxyphenyl)-2-hydroxy-4-methylpentanoic acid (1-oxo-1,3-dihydroisobenzofuran-5-yl)amide;
131) 4-(5-Fluoro-2-methoxyphenyl)-2-hydroxy-2-(2-methoxybenzyl)-4-methylpentanoic acid (1-oxo-1,3-dihydroisobenzofuran-5-yl)amide;
132) 4-(5-Fluoro-2-methoxyphenyl)-2-hydroxy-4-methyl-2-phenethylpentanoic acid (1-oxo-1,3-dihydroisobenzofuran-5-yl)amide;
133) 2-(2-Chlorobenzyl)-4-(5-fluoro-2-methoxyphenyl)-2-hydroxy-4-methylpentanoic acid (1-oxo-1,3-dihydroisobenzofuran-5-yl)amide;
134) 4-(5-Fluoro-2-hydroxyphenyl)-2-hydroxy-4-methyl-2-phenethylpentanoic acid (1-oxo-1,3-dihydroisobenzofuran-5-yl)amide;
135) 4-(5-Fluoro-2-hydroxyphenyl)-2-hydroxy-2-[2-(4-hydroxyphenyl)ethyl]-4-methylpentanoic acid (1-oxo-1,3-dihydroisobenzofuran-5-yl)amide;
136) 2-(2-Chlorobenzyl)-4-(5-fluoro-2-hydroxyphenyl)-2-hydroxy-4-methylpentanoic acid (1-oxo-1,3-dihydroisobenzofuran-5-yl)amide;
137) 4-(5-Fluoro-2-hydroxyphenyl)-2-hydroxy-2-(2-hydroxybenzyl)-4-methylpentanoic acid (1-oxo-1,3-dihydroisobenzofuran-5-yl)amide;
138) 2-(2-Bromobenzyl)-4-(5-fluoro-2-methoxyphenyl)-2-hydroxy-4-methylpentanoic acid (1-oxo-1,3-dihydroisobenzofuran-5-yl)amide;
139) 2-(2-Bromobenzyl)-4-(5-fluoro-2-hydroxyphenyl)-2-hydroxy-4-methylpentanoic acid (1-oxo-1,3-dihydroisobenzofuran-5-yl)amide;
140) 2-Cyclohexylmethyl-4-(5-fluoro-2-methoxyphenyl)-2-hydroxy-4-methylpentanoic acid (3,5-bistrifluoromethylphenyl)amide;
141) 2-Hydroxy-2-(2-methoxybenzyl)-4-methyl-4-phenylpentanoic acid (1-oxo-1,3-dihydroisobenzofuran-5-yl)amide;
142) 2-Hydroxy-2-(2-hydroxybenzyl)-4-methyl-4-phenylpentanoic acid (1-oxo-1,3-dihydroisobenzofuran-5-yl)amide;
143) 5-[2-Benzyl-4-(5-fluoro-2-methoxyphenyl)-2-hydroxy-4-methylpentylamino]-3H-isobenzofuran-1-one;
144) 2-Benzyl-4-(5-fluoro-2-methoxyphenyl)-2-hydroxy-4-methylpentanoic acid (4-cyano-3-trifluoromethylphenyl)amide;
145) 2-Benzyl-4-(5-fluoro-2-hydroxyphenyl)-2-hydroxy-4-methylpentanoic acid (4-cyano-3-trifluoromethylphenyl)amide;
146) 4-(5-Fluoro-2-methoxyphenyl)-2-hydroxy-4-methyl-2-(1-phenylvinyl)pentanoic acid (1-oxo-1,3-dihydroisobenzofuran-5-yl)amide;
147) 4-(5-Fluoro-2-hydroxyphenyl)-2-hydroxy-4-methyl-2-(1-phenylethyl)pentanoic acid (1-oxo-1,3-dihydroisobenzofuran-5-yl)amide;
148) 2-Benzyl-2-hydroxy-4-methyl-4-phenylpentanoic acid (4-methyl-1-oxo-1H-benzo[d][1,2]oxazin-6-yl)amide;
149) 2-Cyclohexylmethyl-2-hydroxy-4-methyl-4-phenylpentanoic acid (4-methyl-1-oxo-1H-benzo[d][1,2]oxazin-6-yl)amide;
150) 2-Cyclohexylmethyl-4-(5-fluoro-2-methoxyphenyl)-2-hydroxy-4-methylpentanoic acid (4-cyano-3-trifluoromethylphenyl)amide;
151) 2-Cyclopentyl-4-(5-fluoro-2-methoxyphenyl)-2-hydroxy-4-methylpentanoic acid (1-oxo-1,3-dihydroisobenzofuran-5-yl)amide;
152) 2-Cyclopentyl-4-(5-fluoro-2-hydroxyphenyl)-2-hydroxy-4-methylpentanoic acid (1-oxo-1,3-dihydroisobenzofuran-5-yl)amide; and
153) 2-Cyclopentylmethyl-4-(5-fluoro-2-hydroxyphenyl)-2-hydroxy-4-methylpentanoic acid (1-oxo-1,3-dihydroisobenzofuran-5-yl)amide,
154) 2-Benzyl-2-hydroxy-4-methyl-4-phenylpentanoic acid (1-oxo-1,3-dihydroisobenzofuran-5-yl)amide
155) 2-Cyclohexylmethyl-2-hydroxy-4-methyl-4-phenylpentanoic acid (1-oxo-1,3-dihydroisobenzofuran-5-yl)amide;
156) 2-Hydroxy-4-methyl-2-(2-methyl-2-phenylpropyl)-4-phenylpentanoic acid (1-oxo-1,3-dihydroisobenzofuran-5-yl)amide;
157) 2-Benzyl-4-(5-fluoro-2-methoxyphenyl)-2-hydroxy-4-methylpentanoic acid (1-oxo-1,3-dihydroisobenzofuran-5-yl)amide;
158) 2-Cyclohexylmethyl-4-(5-fluoro-2-methoxyphenyl)-2-hydroxy-4-methylpentanoic acid (1-oxo-1,3-dihydroisobenzofuran-5-yl)amide;
159) 2-Cyclohexylmethyl-4-(5-fluoro-2-hydroxyphenyl)-2-hydroxy-4-methylpentanoic acid (1-oxo-1,3-dihydroisobenzofuran-5-yl)amide;
160) 2-(3-Fluorobenzyl)-4-(5-fluoro-2-methoxyphenyl)-2-hydroxy-4-methylpentanoic acid (1-oxo-1,3-dihydroisobenzofuran-5-yl)amide;
161) 2-(2-Fluorobenzyl)-4-(5-fluoro-2-methoxyphenyl)-2-hydroxy-4-methylpentanoic acid (1-oxo-1,3-dihydroisobenzofuran-5-yl)amide;
162) 2-(2-Fluorobenzyl)-4-(5-fluoro-2-hydroxyphenyl)-2-hydroxy-4-methylpentanoic acid (1-oxo-1,3-dihydroisobenzofuran-5-yl)amide;
163) 2-(3,4-Difluorobenzyl)-4-(5-fluoro-2-hydroxyphenyl)-2-hydroxy-4-methylpentanoic acid (1-oxo-1,3-dihydroisobenzofuran-5-yl)amide;
164) 2-(4-Fluorobenzyl)-4-(5-fluoro-2-hydroxyphenyl)-2-hydroxy-4-methylpentanoic acid (1-oxo-1,3-dihydroisobenzofuran-5-yl)amide;
165) 2-Benzyl-4-(5-fluoro-2-methoxyphenyl)-2-hydroxy-4-methylpentanoic acid (4-methyl-1-oxo-1H-benzo[d][1,2]oxazin-6-yl)amide;
166) 2-Benzyl-4-(5-fluoro-2-hydroxyphenyl)-2-hydroxy-4-methylpentanoic acid (4-methyl-1-oxo-1H-benzo[d][1,2]oxazin-6-yl)amide;
167) 2-Cyclohexylmethyl-4-(5-fluoro-2-methoxyphenyl)-2-hydroxy-4-methylpentanoic acid (4-methyl-1-oxo-1H-benzo[d][1,2]oxazin-6-yl)amide;
168) 4-(5-Fluoro-2-methoxyphenyl)-2-hydroxy-4-methyl-2-(2-methylbenzyl)pentanoic acid (1-oxo-1,3-dihydroisobenzofuran-5-yl)amide;
169) 4-(5-Fluoro-2-hydroxyphenyl)-2-hydroxy-4-methyl-2-(2-methylbenzyl)pentanoic acid (1-oxo-1,3-dihydroisobenzofuran-5-yl)amide;
170) 2-Cyclohexylmethyl-4-(5-fluoro-2-hydroxyphenyl)-2-hydroxy-4-methylpentanoic acid (4-methyl-1-oxo-1H-benzo[d][1,2]oxazin-6-yl)amide;
171) 2-(2-Chlorobenzyl)-4-(5-fluoro-2-methoxyphenyl)-2-hydroxy-4-methylpentanoic acid (1-oxo-1,3-dihydroisobenzofuran-5-yl)amide;
172) 4-(5-Fluoro-2-hydroxyphenyl)-2-hydroxy-4-methyl-2-phenethylpentanoic acid (1-oxo-1,3-dihydroisobenzofuran-5-yl)amide;
173) 2-(2-Chlorobenzyl)-4-(5-fluoro-2-hydroxyphenyl)-2-hydroxy-4-methylpentanoic acid (1-oxo-1,3-dihydroisobenzofuran-5-yl)amide;
174) 4-(5-Fluoro-2-hydroxyphenyl)-2-hydroxy-2-(2-hydroxybenzyl)-4-methylpentanoic acid (1-oxo-1,3-dihydroisobenzofuran-5-yl)amide;
175) 2-(2-Bromobenzyl)-4-(5-fluoro-2-methoxyphenyl)-2-hydroxy-4-methylpentanoic acid (1-oxo-1,3-dihydroisobenzofuran-5-yl)amide;
176) 2-(2-Bromobenzyl)-4-(5-fluoro-2-hydroxyphenyl)-2-hydroxy-4-methylpentanoic acid (1-oxo-1,3-dihydroisobenzofuran-5-yl)amide;
177) 5-[2-Benzyl-4-(5-fluoro-2-methoxyphenyl)-2-hydroxy-4-methylpentylamino]-3H-isobenzofuran-1-one;
178) 4-(5-Fluoro-2-hydroxyphenyl)-2-hydroxy-4-methyl-2-(1-phenylethyl)pentanoic acid (1-oxo-1,3-dihydroisobenzofuran-5-yl)amide;
179) 2-Benzyl-2-hydroxy-4-methyl-4-phenylpentanoic acid (4-methyl-1-oxo-1H-benzo[d][11,2]oxazin-6-yl)amide;
180) 2-Cyclohexylmethyl-2-hydroxy-4-methyl-4-phenylpentanoic acid (4-methyl-1-oxo-1H-benzo[d][1,2]oxazin-6-yl)amide;
181) 2-Cyclopentyl-4-(5-fluoro-2-hydroxyphenyl)-2-hydroxy-4-methylpentanoic acid (1-oxo-1,3-dihydroisobenzofuran-5-yl)amide; and
182) 2-Cyclopentylmethyl-4-(5-fluoro-2-hydroxyphenyl)-2-hydroxy-4-methylpentanoic acid (1-oxo-1,3-dihydroisobenzofuran-5-yl)amide.

22. Use of the compounds of the general formula (I) for producing a medicament for oral administration comprising at least one compound of the general formula (I) and, where appropriate, at least one further active ingredient together with pharmaceutically acceptable excipients and/or carriers.

23. Use of the compounds of the general formula (I) according to claim 22, where the further active ingredient is a SERM (selective estrogen receptor modulator), an aromatase inhibitor, antiestrogen or a prostaglandin.

24. Use of the compounds of the general formula (I) according to claim 23, where the active ingredients may be tamoxifen, 5-(4-{5-[(RS)-(4,4,5,5,5-pentafluoropentyl)-sulphynyl]pentyloxy}phenyl)-6-phenyl-8,9-dihydro-7H-benzocyclohepten-2-ol, ICI 182 780 (7alpha-[9-(4,4,5,5-pentafluoropentylsulphynyl)nonyl]estra-1,3,5(10)-triene-3,17beta-diol), 11beta-fluoro-7alpha-[5-(methyl {3-[(4,4,5,5,5-pentafluoropentyl)-sulphanyl]propyl}amino)pentyl]estra-1,3,5(10)-triene-3,17beta-diol, 11beta-fluoro-7alpha-{5-[methyl(7,7,8,8,9,9,10,10,10-nonafluorodecyl)amino]pentyl}estra-1,3,5(10)-triene-3,17beta-diol, 11beta-fluoro-17alpha-methyl-7alpha-{5-[methyl(8,8,9,9,9-pentafluorononyl)amino]pentyl}estra-1,3,5(10)-triene-3,17beta-diol, clomifen, raloxifen, fadrozole, formestane, letrozole, anastrozole or atamestane.
